(19) United States
(12) Patent Application Publication
(10) Pub. No.: US 2005/0180952 A1
(43) Pub. Date: Aug. 18, 2005

(54) Methods for intradermal delivery of therapeutics agents

(76) Inventors: Ronald J. Pettis, Cary, NC (US); Alfred J. Harvey, Durham, NC (US); Robert L. Campbell, Bahama, NC (US); John A. Mikszta, Durham, NC (US); John M. Brittingham, Wake Forest, NC (US); Glenn P. Vonk, Fuquay Varina, NC (US); Colleen M. Nycz, Raleigh, NC (US)

Correspondence Address: JONES DAY, 222 EAST 41ST ST, NEW YORK, NY, 10017, US

(21) Appl. No.: 10/868,482

(22) Filed: Jun. 14, 2004

Related U.S. Application Data
(60) Provisional application No. 60/550,197, filed on Mar. 3, 2004.
(60) Provisional application No. 60/497,702, filed on Aug. 26, 2003.

(51) Int. Cl. A61K 38/20; A61K 38/21; A61K 39/395; A61K 45/00

(52) U.S. Cl. 424/85.2; 424/85.4; 424/130.1; 514/2; 604/500

(57) ABSTRACT

The present invention relates to methods and devices for delivering one or more biologically active agents, particularly therapeutic agents, to the intradermal compartment of a subject's skin. The present invention provides an improved method of delivery of biologically active agents, such as therapeutic agents, through lymphatic vasculature accessed by intradermal delivery. Therapeutic agents to be delivered in accordance with the present invention include, but are not limited to, antineoplastic agents, chemotherapeutic agents, antibodies, antibiotics, anti-angiogenesis agents, anti-inflammatory agents, and immunotherapeutic agents. Therapeutic agents delivered in accordance with the present invention have improved bioavailability, including improved systemic distribution and improved delivery to particular tissues. Therapeutic agents delivered in accordance with the methods of the invention have an improved clinical utility and therapeutic efficacy relative to other drug delivery methods, including intraperitoneal, intramuscular and subcutaneous delivery. The methods of the present invention provide benefits and improvements over conventional drug delivery methods including dose sparing, increased drug efficacy, reduced side effects, reduced metastatic potential and prolonged survival.

This application claims priority to U.S. Provisional Application No. 60/550,197 filed on Mar. 3, 2004; and U.S. Provisional Application No. 60/497,702, filed Aug. 26, 2003, each of which is incorporated herein by reference in its entireties.

### 1. FIELD OF THE INVENTION

The present invention relates to methods and devices for delivering one or more biologically active agents, particularly therapeutic agents, to the intradermal compartment of a subject's skin. The present invention provides an improved method of delivery of biologically active agents, such as therapeutic agents, through lymphatic vasculature accessed by intradermal delivery. Therapeutic agents to be delivered in accordance with the present invention include, but are not limited to, antineoplastic agents, chemotherapeutic agents, antibodies, antibiotics, anti-angiogenesis agents, anti-inflammatory agents, and immunotherapeutic agents. Therapeutic agents delivered in accordance with the present invention have improved bioavailability, including improved systemic distribution and improved delivery to particular tissues. Therapeutic agents delivered in accordance with the methods of the invention have an improved clinical utility and therapeutic efficacy relative to other drug delivery methods, including intraperitoneal, intramuscular and subcutaneous delivery. The methods of the present invention provide benefits and improvements over conventional drug delivery methods including dose sparing, increased drug efficacy, reduced side effects, reduced metastatic potential and prolonged survival.

### 2. BACKGROUND OF THE INVENTION

The importance of efficiently and safely administering pharmaceutical agents such as therapeutic agents and drugs has long been recognized. Difficulties associated with ensuring adequate bioavailability and reproducible absorption of large molecules, such as proteins that have arisen out of the biotechnology industry, have been recently highlighted (Cleland et al., Curr. Opin. Biotechnol. 12: 212-219, 2001). The use of conventional needles has long provided one approach for delivering pharmaceutical agents to humans and animals by administration through the skin. In general, injection avoids harsh conditions associated with oral delivery that commonly mitigate the desired effects of most biological therapies. Injection may also provide faster therapeutic effect than oral administration. Considerable effort has been made to achieve reproducible and efficacious delivery needle-based injection while improving the ease of use and reducing patient apprehension and/or pain associated with conventional needles. Furthermore, certain transcutaneous delivery systems eliminate needles entirely, and rely upon simple hydrophobic adsorption, chemical mediators or external driving forces such as iontophoretic currents or electroporation or thermal poration or sonophoresis to breach the stratum corneum (the outermost layer of the skin) and deliver agents through the surface of the skin. However, such delivery systems do not, in general, reproducibly traverse the skin barriers or deliver pharmaceutical agents to a given depth below the surface of the skin. Consequently, clinical results can be variable. Thus, mechanical breach of the stratum corneum, such as with needles, is believed to provide the most reproducible method of administration of agents through the surface of the skin, and provides control and reliability in the placement of the administered agents.

Approaches for delivering agents beneath the surface of the skin have almost exclusively involved transdermal injections or infusions, i.e. delivery of agents through the skin to a site beneath the skin. Transdermal injections and infusions include subcutaneous, intramuscular or intravenous routes of administration of which, intramuscular (IM) and subcutaneous (SC) injections have been the most commonly used.

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University Press, New York, 1991). The epidermis is subdivided into five layers or strata of a total thickness of between 75 and 150 μm. Beneath the epidermis lies the dermis, which contains two layers, an outermost portion referred to as the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue.

As noted above, both the subcutaneous tissue and muscle tissue have been commonly used as sites for administration of pharmaceutical agents, including therapeutic agents. The dermis, however, has rarely been targeted as a site for administration of agents, and this may be due, at least in part, to the difficulty of precise needle placement into the intradermal compartment. Furthermore, even though the dermis, in particular, the papillary dermis has been known to have a high degree of vascularity, it has not heretofore been appreciated that one could take advantage of this high degree of vascularity to obtain an improved absorption profile for administered agents compared to subcutaneous administration.

One approach to administration beneath the surface to the skin and into the region of the intradermal compartment has been routinely used in the Mantoux tuberculin test. In this procedure, a purified protein derivative is injected at a shallow angle to the skin surface using a 27 or 30 gauge needle (Flynn et al., Chest 106: 1463-5, 1994). A degree of uncertainty in placement of the injection can, however, result in some false negative test results. Moreover, the test has involved a localized injection to elicit a response at the site of injection and the Mantoux approach has not led to the use of intradermal injection for systemic administration of agents.

Some groups have reported on systemic administration by what has been characterized as “intradermal” injection. In one such report, a comparison study of subcutaneous and what was described as “intradermal” injection was performed (Autret et al., Therapie 46:5-8, 1991). The pharmaceutical agent tested was calcitonin, a protein of a molecular weight of about 3600. Although it was stated that the drug was injected intradermally, the injections used a 4 mm needle pushed up to the base at an angle of 60. This would have resulted in placement of the injectate at a depth of about 3.5 mm and into the lower portion of the reticular dermis or into the subcutaneous tissue rather than into the vascularized papillary dermis. If, in fact, this group injected into the lower portion of the reticular dermis rather than into the subcutaneous tissue, it would be expected that the agent would either be slowly absorbed in the relatively less vascular reticular dermis or diffuse into the subcutaneous region to result in what would be functionally the same as subcutaneous administration and absorption. Such actual or functional subcutaneous administration would explain the reported lack of difference between subcutaneous and what was characterized as intradermal administration, in the times at which maximum plasma concentration was reached, the concentrations at each assay time and the areas under the curves.

Similarly, Bressolle et al., administered sodium ceftazidime in what was characterized as “intradermal” injection using a 4 mm needle (Bressolle et al., J. Pharm. Sci. 82:1175-1178, 1993). This would have resulted in injection to a dept of 4 mm below the skin surface to produce actual or functional subcutaneous injection, although good subcutaneous absorption would have been anticipated in this instance because sodium ceftazidime is hydrophilic and of relatively low molecular weight.

Another group reported on what was described as intradermal drug delivery device (U.S. Pat. No. 5,007,501). Injection was indicated to be at a slow rate and the injection site was intended to be in some region below the epidermis, i.e., the interface between the epidermis and the dermis or the interior of the dermis or subcutaneous tissue. This reference, however, provided no teachings that would suggest a selective administration into the dermis nor did the reference suggest any possible pharmacokinetic advantage that might result from such selective administration.

Thus, there remains a continuing need for efficient and safe methods and devices for administration of pharmaceutical agents, especially therapeutic agents.

### 3. SUMMARY OF THE INVENTION

The present invention provides a method for administering one or more biologically active agents, preferably therapeutic agents, to a subject's skin, in which the biologically active agent is delivered to the intradermal compartment of the subject's skin. In a preferred embodiment, the therapeutic agents to be delivered in accordance with the invention include, but are not limited to, antineoplastic agents, chemotherapeutic agents, antibodies, antibiotics, anti-angiogenesis agents, anti-inflammatory agents, and immunotherapeutic agents.

The present invention is based, in part, on the inventors' discovery that when agents are delivered to the intradermal compartment, they are rapidly transported to the local lymphatic system, systemically distributed and distributed to deeper tissues. Intradermal delivery of therapeutic agents in accordance with the present invention can directly access both the venous and lymphatic networks of the dermis and provide unique systemic pharmacokinetic outcomes. By accessing these networks, therapeutic advantages can be achieved, including but not limited to, improved clinical utility and therapeutic efficacy. Specifically, the inventors have found that administration of therapeutic agents to the intradermal compartment results in an enhanced therapeutic effect relative to other drug delivery methods, including intraperitoneal delivery.

In a particular embodiment, the present invention provides an improved method for delivery of therapeutic agents, including, but not limited to, antineoplastic agents, chemotherapeutic agents, antibodies, antibiotics, anti-angiogenesis agents, anti-inflammatory agents, immunotherapeutic agents, and anti-viral agents with improved clinical utility and therapeutic efficacy.

Biologically active agents, such as therapeutic agents, administered in accordance with the methods of the invention are rapidly transported through both the venous and lymphatic networks of the dermis. Therapeutic agents delivered in accordance with the methods of the invention are deposited in the intradermal compartment and distributed with high bioavailability to the lymphatic tissue local to the administration site, followed by a more wide spread lymphatic delivery in to the general circulation. This method of delivery of therapeutic agents is particularly useful for treating disorders which utilize both networks, including, but not limited to, cancer tumor growth and metastasis, viral infection, bacterial infection, parasitic infection, immune disorders and metabolic disorders.

Intradermal delivery of therapeutic agents in accordance with the present invention can directly access both the venous and lymphatic networks of the dermis and provide unique systemic phamacokinetic outcomes. By accessing these networks in the vicinity of the target, such as the tumor, therapeutic advantages may be achieved. The intradermally administered therapeutic agents may also access the immune cells mediated by the lymphatic network. Further, intradermally delivered therapeutic agents will result in systemic distribution providing the added benefit of reaching extensively disseminated sites and organs, in a manner similar to many current therapies.

The present invention provides an improved method of enhancing the bioavailability of a biologically active agent to a particular tissue, including but not limited to skin tissue, lymphatic tissue (e.g., lymph nodes), mucosal tissue, reproductive tissue, cervical tissue, vaginal tissue and any part of the body that consists of different types of tissue and that performs a particular function, i.e., an organ, including but not limited to lung, spleen, colon, thymus. In some embodiments, the tissue includes any tissue that interacts with or is accessible to the environment, e.g., skin, mucosal tissue. Other tissues encompassed by the invention include without limitation Haemolymphoid System; Lymphoid Tissue (e.g., Epithelium-associated lymphoid Tissue and Mucosa-associated lymphoid Tissue or MALT (MALT can be further divided as organized mucosa-associated lymphoid Tissue (O-MALT) and diffused lymphoid tissue (D-MALT)); primary Lymphoid Tissue (e.g., thymus and bone marrow); Secondary Lymphoid Tissue (e.g., lymph node, spleen, alimentary, respiratory and Urigenital). It will be appreciated by one skilled in the art that MALT secondary includes gut associated lymphoid tissue (GALT); Bronchial associated lymphoid tissue (BALT), and genitourinary system. MALT specifically comprises lymph nodes, spleen, tissue associated with epithelial surfaces such as palentine and nasopharyngeal tonsils, Peyer's Patches in the small intestine and various nodules in the respiratory and urinogenital systems, the skin and conjunctivia of the eye. O-MALT includes the peripharyngeal lymphoid ring of the tonsils (palentine, lingual, nasopharyngeal and tubal), Oesophageal nodules and similar lymphoid tissue scattered throughout the alimentary tract from the duuuodenum to the anal canal.

Intradermal delivery of biologically active agents in accordance with the present invention provides among other benefits, rapid uptake into local lymphatics, improved targeting and deposition of the delivered agent to a particular tissue, and improved systemic and tissue bioavailability. Such benefits are especially useful for the delivery of therapeutic agents, such as antineoplastic agents, antibodies and antibiotics. Intradermal delivery of agents in accordance with the methods of the invention deposits the agent into the intradermal and lymphatic compartments and deeper tissues, resulting in rapid and biologically significant concentrations of the agents in these compartments and tissues.

By direct lymphatic targeting of various therapeutic agents using intradermal delivery beneficial therapeutic outcomes are realized, including dose sparing, increased drug efficacy, reduced side effects, reduced metastatic potential, and prolonged survival. The present invention provides improved methods for treating diseases in that the delivery methods of the invention allow for both systemic and localized deposition of therapeutic agents. As a result, the present invention provides improved methods for treatment of a diseases such as cancer, by improving the amount of the agent deposited, tissue bioavailability, faster onset and clearance of the delivered therapeutic agent. The invention provides a method for administration of at least one therapeutic agent for the treatment of a disease, particularly cancer, comprising delivering the agent into the intradermal compartment of a subject's skin at a controlled rate, volume and pressure so that the agent is deposited into the ID compartment and taken up by the lymphatic vasculature.

The present invention also provides improved methods for treatment of a disease that is localized to particular tissues and organs of the body, such as infection of those tissues and organs, e.g., respiratory infection, by improving the amount of the agent deposited, tissue bioavailability, faster onset and clearance of the delivered therapeutic agent. The invention provides a method for administration of at least one therapeutic agent for the treatment of a disease, particularly infection, comprising delivering the agent into the intradermal compartment of a subject's skin at a controlled rate, volume and pressure so that the agent is deposited into the intradermal compartment and taken up by the lymphatic vasculature.

As used herein, delivery to the intradermal compartment or intradermally delivered is intended to mean administration of a biologically active agent into the dermis in such a manner that the agent readily reaches the richly vascularized papillary dermis and is rapidly absorbed into the blood capillaries and/or lymphatic vessels to become systemically bioavailable. Such can result from placement of the agent in the upper region of the dermis, i.e., the papillary dermis or in the upper portion of the relatively less vascular reticular dermis such that the agent readily diffuses into the papillary dermis. The controlled delivery of a biologically active agent in this dermal compartment below the papillary dermis in the reticular dermis, but sufficiently above the interface between the dermis and the subcutaneous tissue, should enable an efficient (outward) migration of the agent to the (undisturbed) vascular and lymphatic microcapillary bed (in the papillary dermis), where it can be absorbed into systemic circulation via these microcapillaries without being sequestered in transit by any other cutaneous tissue compartment. In some embodiments, placement of a biologically active agent predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption of the agent. Although not intending to be bound by a particular mechanism of action, placement of the biologically active agent predominately at greater depths and/or into the lower portion of the reticular dermis may result in less effective uptake of the agent by the lymphatics, as the agent will be slowly absorbed in the less vascular reticular dermis or in the subcutaneous compartment.

The improved benefits associated with ID delivery of biologically active agents in accordance with the methods of the invention can be achieved using not only microdevice-based injection systems, but other delivery systems such as needle-less or needle-free ballistic injection of fluids or powders into the ID compartment, enhanced ionotophoresis through microdevices, and direct deposition of fluid, solids, or other dosing forms into the skin. In specific embodiments, the administration of the biologically active agent is accomplished through insertion of a needle or cannula into the intradermal compartment of the subject's skin.

3.1 Definitions

As used herein, “intradermal” refers to administration of a biologically active agent into the dermis in such a manner that the agent readily reaches the richly vascularized papillary dermis and is rapidly absorbed into the blood capillaries and/or lymphatic vessels to become systemically bioavailable. Such can result from placement of the agent in the upper region of the dermis, i.e., the papillary dermis or in the upper portion of the relatively less vascular reticular dermis such that the agent readily diffuses into the papillary dermis. The controlled delivery of a biologically active agent in this dermal compartment below the papillary dermis in the reticular dermis, but sufficiently above the interface between the dermis and the subcutaneous tissue, should enable an efficient (outward) migration of the agent to the (undisturbed) vascular and lymphatic microcapillary bed (in the papillary dermis), where it can be absorbed into systemic circulation via these microcapillaries without being sequestered in transit by any other cutaneous tissue compartment. In some embodiments, placement of a biologically active agent predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption the agent. Although not intending to be bound by a particular mechanism of action, placement of the biologically active agent predominately at greater depths and/or into the lower portion of the reticular dermis or the SC compartment which results in less effective uptake by the lymphatics.

As used herein, “intradermal delivery” means the delivery of agents to the intradermal compartment as described by Pettis et al. in WO 02/02179 A1 (PCT/JUS01/20782) and U.S. application Ser. No. 09/606,909; each of which is incorporated herein by reference in their entireties.

As used herein subcutaneous delivery refers to deposition of an agent into the subcutaneous layer of a subject's skin at a depth greater than 2.5 mm.

As used herein, “pharmacokinetics, pharmacodynamics and bioavailability” are as described by Pettis et al. in WO 02/02179 A1 (PCT/US01/20782 having a priority date of Jun. 29, 2000).

As used herein, “improved pharmacokinetics” means increased bioavailability, decreased lag time (T_{lag}), decreased Tₘₐₓ, more rapid absorption rates, more rapid onset and/or increased Cₘₐₓ for a given amount of agent administered, compared to conventional administration methods.

As used herein, “bioavailability”, means the total amount of a given dosage of the administered agent that reaches the blood compartment. This is generally measured as the area under the curve in a plot of concentration vs. time.

As used herein “tissue” refers to a group or layer of cells that together perform a function including but not limited to, skin tissue, lymphatic tissue (e.g., lymph nodes), mucosal tissue, reproductive tissue, cervical tissue, vaginal tissue and any part of the body that consists of different types of tissue and that performs a particular function, i.e., an organ, including but not limited to lung, spleen, colon, thymus. As used herein, tissue includes any tissue that interacts with or is accessible to the environment, e.g., skin, mucosal tissue.

As used herein, “tissue-bioavailability” means the amount of an agent that is biologically available in vivo in a particular tissue. These amounts are commonly measured as activities that may relate to binding, labeling, detection, transport, stability, biological effect, or other measurable properties useful for diagnosis and/or therapy. In addition, it is understood that the definition of “tissue-bioavailability” also includes the amount of an agent available for use in a particular tissue. “Tissue-bioavailability” includes the total amount of the agent accumulated in a particular tissue, the amount of the agent presented to the particular tissue, the amount of the agent accumulated per mass/volume of particular tissue, and amount of the agent accumulated per unit time in a particular mass/volume of the particular tissue. Tissue bioavailability includes the amount of an agent that is available in vivo in a particular tissue or a collection of tissues such as those that make up the vasculature and/or various organs of the body (ie., a part of the body that consists of different types of tissue and that performs a particular function. Examples include the spleen, thymus, lung, lymph nodes, heart and brain).

As used herein, “lag time,” means the delay between the administration of the agent and time to measurable or detectable blood or plasma levels. Tₘₐₓ is a value representing the time to achieve maximal blood concentration of the agent, and Cₘₐₓ is the maximum blood concentration reached with a given dose and administration method. The time for onset is a function of T_{lag}, Tₘₐₓ and Cₘₐₓ, as all of these parameters influence the time necessary to achieve a blood (or target tissue) concentration necessary to realize a biological effect. Tₘₐₓ and Cₘₐₓ can be determined by visual inspection of graphical results and can often provide sufficient information to compare two methods of administration of a agent. However, numerical values can be determined more precisely by kinetic analysis using mathematical models and/or other means known to those of skill in the art.

As used herein, “conventional delivery” means any method for delivering any material that has, or is thought to have, improved biological kinetics and biological dynamics similar to, or slower than, subcutaneous delivery. Conventional delivery may include subcutaneous, iontophoretic, and intradermal delivery methods such as those described in U.S. Pat. No. 5,800,420 to Gross.

As used herein, the terms “disorder” and “disease” are used interchangeably to refer to a condition in a subject. Diseases include to any interruption, cessation, or disorder of body functions, systems or organs.

As used herein, the term “cancer” refers to a neoplasm or tumor resulting from abnormal uncontrolled growth of cells. As used herein, cancer explicitly includes, leukemias and lymphomas. The term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or weblike matrices in a three-dimensional basement membrane or extracellular matrix preparation. Non-cancer cells do not form colonies in soft agar and form distinct sphere-like structures in three-dimensional basement membrane or extracellular matrix preparations. Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless explicative potential, and sustained angiogenesis. The term “cancer cell” is meant to encompass both pre-malignant and malignant cancer cells. In some embodiments, cancer refers to a benign tumor, which has remained localized. In other embodiments, cancer refers to a malignant tumor, which has invaded and destroyed neighboring body structures and spread to distant sites. In yet other embodiments, the cancer is associated with a specific cancer antigen.

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., delay or minimize the spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the terms “prophylactic agent” and “prophylactic agents” refer to any agent(s) which can be used in the prevention of a disorder, or prevention of recurrence or spread of a disorder.

As used herein, a “prophylactically effective amount” may refer to the amount of prophylactic agent sufficient to prevent the recurrence or spread of hyperproliferative disease, particularly cancer, or the occurrence of such in a patient, including but not limited to those predisposed to hyperproliferative disease, for example those genetically predisposed to cancer or previously exposed to carcinogens. A prophylactically effective amount may also refer to the amount of the prophylactic agent that provides a prophylactic benefit in the prevention of disease. Further, a prophylactically effective amount with respect to a prophylactic agent of the invention means that amount of prophylactic agent alone, or in combination with other agents, that provides a prophylactic benefit in the prevention of disease.

As used herein, the terms “treat,” “treating” and “treatment” refer to the eradication, reduction or amelioration of symptoms of a disease or disorder. In some embodiments, treatment refers to the eradication, removal, modification, or control of primary, regional, or metastatic cancer tissue that results from the administration of one or more therapeutic agents. In certain embodiments, such terms refer to the minimizing or delaying the spread of cancer resulting from the administration of one or more therapeutic agents to a subject with such a disease.

As used herein, the terms “manage,” “managing” and “management” refer to the beneficial effects that a subject derives from administration of a prophylactic or therapeutic agent, which does not result in a cure of the disease. In certain embodiments, a subject is administered one or more prophylactic or therapeutic agents to “manage” a disease so as to prevent the progression or worsening of the disease.

As used herein, the terms “prevent”, “preventing” and “prevention” refer to the prevention of the recurrence or onset of one or more symptoms of a disorder in a subject resulting from the administration of a prophylactic or therapeutic agent

As used herein, the phrase “side effects” encompasses unwanted and adverse effects of a prophylactic or therapeutic agent. Adverse effects are always unwanted, but unwanted effects are not necessarily adverse. An adverse effect from a prophylactic or therapeutic agent might be harmful or uncomfortable or risky. Side effects from chemotherapy include, but are not limited to, gastrointestinal toxicity such as, but not limited to, early and late-forming diarrhea and flatulence, nausea, vomiting, anorexia, leukopenia, anemia, neutropenia, asthenia, abdominal cramping, fever, pain, loss of body weight, dehydration, alopecia, dyspnea, insomnia, dizziness, mucositis, xerostomia, and kidney failure, as well as constipation, nerve and muscle effects, temporary or permanent damage to kidneys and bladder, flu-like symptoms, fluid retention, and temporary or permanent infertility. Side effects from radiation therapy include but are not limited to fatigue, dry mouth, and loss of appetite. Side effects from biological therapies/immunotherapies include but are not limited to rashes or swellings at the site of administration, flu-like symptoms such as fever, chills and fatigue, digestive tract problems and allergic reactions. Side effects from hormonal therapies include but are not limited to nausea, fertility problems, depression, loss of appetite, eye problems, headache, and weight fluctuation. Additional undesired effects typically experienced by patients are numerous and known in the art, see, e.g., the Physicians' Desk Reference (56^{th} ed., 2002), which is incorporated herein by reference in its entirety.

### 4. DESCRIPTION OF THE FIGURES

FIG. 1 illustrates effects of routes of IL-12 administration on inhibition of tumor growth by IL-12.

FIG. 2 illustrates effects of routes of IL-12 administration on mortality of tumor-containing mice.

FIG. 3 illustrates effects of routes of IL-12 administration on percent NK cells detected in DLN as determined by FACS analysis.

FIG. 4 illustrates lung levels of RSV-Specific Antibody after ID and IM Delivery. This area graph shows the amount of antibody detected in lung tissue collected from animals at hours 3 and 24, weeks 1, 2, 3 and 4.

FIG. 5 illustrates the determination of the optimal IN delivery volume and lung harvest time in Balb/c Model.

FIG. 6 illustrates two possible bioavailability outcomes by splitting the dose.

FIG. 7 illustrates synagis in lung tissue lysates.

FIG. 8 shows the results from synagis plaque assay.

FIG. 9 illustrates an exploded, perspective illustration of a needle assembly designed according to this invention.

FIG. 10 illustrates a partial cross-sectional illustration of the embodiment in FIG. 9.

FIG. 11 illustrates Embodiment of FIG. 9 attached to a syringe body to form an injection device.

### 5. DESCRIPTION DESCRIPTION OF THE INVENTION

The present invention provides a method for administering one or more biologically active agents, preferably a therapeutic agent, to a subject's skin, in which the biologically active agent is delivered to the intradermal compartment of the subject's skin. The present invention is based, in part, on the unexpected discovery by the inventors that when such therapeutic agents are delivered to the intradermal compartment, they are transported to the local lymphatic system rapidly compared to conventional modes of delivery, including subcutaneous delivery and intramuscular delivery, and thus provide the benefits disclosed herein. Although not intending to be bound by a particular mechanism of action, therapeutic agents delivered in accordance with the methods of the invention are transported in vivo through the local lymphatic system, into the systemic blood circulation and into deeper tissue environments.

The present invention provides an improved method of delivery of biologically active agents in that it provides among other benefits, rapid uptake into the local lymphatics, improved targeting to a particular tissue, i.e., improved deposition of the delivered therapeutic agent into the particular tissue, i.e., group or layer of cells that together perform a specific function, improved systemic bioavailability, improved tissue bioavailability, improved deposition of a pre-selected volume of the agent to be administered, improved tissue-specific kinetics rapid biological and pharmaco-dynamics (PD), and rapid biological and pharmacokinetics (PK). Such benefits of the methods of the invention are especially useful for the delivery of therapeutic agents. Intradermal delivery of a therapeutic agent in accordance with the methods of the invention deposits the therapeutic agent into the intradermal and lymphatic compartments thus creating a rapid and biologically significant concentration of the therapeutic agent in these compartments.

Intradermally delivered therapeutic agents have improved tissue bioavailability in a particular tissue, including but not limited to mucosal layer, skin tissue, lymphatic tissue (e.g., lymph nodes), mucosal tissue, reproductive tissue, cervical tissue, vaginal tissue and any part of the body that consists of different types of tissue and that performs a particular function, i.e., an organ, including but not limited to lung, spleen, colon, thymus. In some embodiments, the tissue includes any tissue that interacts with or is accessible to the environment, e.g., skin, mucosal tissue. Other tissues encompassed by the invention include without limitation Haemolymphoid System; Lymphoid Tissue (e.g., Epithelium-associated lymphoid Tissue and Mucosa-associated lymphoid Tissue or MALT (MALT can be further divided as organized mucosa-associated lymphoid Tissue (O-MALT) and diffused lymphoid tissue (D-MALT)); primary Lymphoid Tissue (e.g., thymus and bone marrow); Secondary Lymphoid Tissue (e.g., lymph node, spleen, alimentary, respiratory and Urigenital). It will be appreciated by one skilled in the art that MALT secondary includes gut associated lymphoid tissue (GALT); Bronchial associated lymphoid tissue (BALT), and genitourinary system. MALT specifically comprises lymph nodes, spleen, tissue associated with epithelial surfaces such as palentine and nasopharyngeal tonsils, Peyer's Patches in the small intestine and various nodules in the respiratory and urinogenital systems, the skin and conjunctivia of the eye. O-MALT includes the peripharyngeal lymphoid ring of the tonsils (palentine, lingual, nasopharyngeal and tubal), Oesophageal nodules and similar lymphoid tissue scattered throughout the alimentary tract from the duuuodenum to the anal canal. tissue and any part of the body that consists of different types of tissue and that performs a particular function, i.e., an organ, including but not limited to lung, spleen, colon, thymus

The delivery of a therapeutic agent in accordance with the methods of the invention results in improved tissue bioavailability as compared to when the same agent is delivered to the subcutaneous (SC) or intramuscular compartment. Improved tissue bioavailability of agents delivered in accordance with the methods of the invention is particularly useful when delivering therapeutic agents, as it results in beneficial therapeutic outcomes, including dose paring, increased drug efficacy, and reduced side effects.

Therapeutic agents delivered in accordance with the methods of the invention are deposited in the intradermal compartment and first distributed with high bioavailability to the lymphatic tissue local to the administration site, followed by a more wide spread lymphatic delivery in to the general circulation. In some embodiments, the methods of the present invention are particularly effective for treatment of a disease, disorder, or infection in deeper tissues.

In some embodiments, the concentration of the biologically active agent deposited in a particular tissue after ID delivery is about 5 nanograms of the agent agent per 50 micrograms of the particular tissue; 10 picograms of the agent per 50 micrograms of the particular tissue; 29 nanograms of the agent per 50 micrograms of the particular tissue; 10 picograms of the agent per 50 micrograms of the particular tissue to about 29 nanograms of the agent per 50 micrograms of the particular tissue; 10 picograms of the agent per 50 micrograms of particular tissue to about 150 nanograms of the agent per 50 micrograms of the particular tissue, or 10 picograms of the agent per 50 micrograms of particular tissue.

The present invention encompasses methods for intradermal delivery of biologically active agents, particularly therapeutic agents such that the agent has a higher tissue bioavailability in a particular tissue as compared to when the agent is delivered by a route other than intradermal delivery such as SC delivery, intramuscular delivery, intravenous delivery, and epidermal delivery. In some embodiments, biologically active agents, particularly therapeutic agents delivered in accordance with the methods of the invention have a similar bioavailability, including tissue bioavailability as compared to when the agent is delivered intravenously.

The present invention encompasses methods for intradermal delivery of biologically active agents, particularly therapeutic agents such that the agent has a higher tissue bioavailability in a particular tissue as compared to when the agent is delivered to a deeper tissue compartment, e.g., SC. Preferably, the biologically active agent delivered in accordance with the methods of the invention is a therapeutic agent, administered for the treatment, prevention, delay of onset or the progression, or management of a disease including but not limited to cancer (e.g., lymphoma, leukemia, breast cancer, melanoma, lung cancer, renal cancer, and colorectal cancer), metastasis, tumor growth, or an infectious disease.

The present invention encompasses methods for intradermal delivery of biologically active agents, particularly therapeutic agents such that the agent has higher therapeutic efficacy as compared to when the agent is delivered to a deeper tissue compartment, e.g., SC. In some embodiments, biologically active agents, particularly therapeutic agents delivered in accordance with the methods of the invention have a similar therapeutic efficacy as compared to when the agent is delivered intravenously.

The invention encompasses methods of treating, preventing or management of a disease comprising administering at least one therapeutic agent at a pre-selected dose, wherein the pre-selected dose is reduced by at least half a fold, at least 5 fold, at least 10 fold compared to the dose of the agent that is conventionally delivered by other routes of delivery such as SC, IM, and IV.

In some embodiments, the invention encompasses methods of treating, preventing, or managing cancer, cancer metastasis or tumor growth in a human subject in need thereof, comprising delivering a therapeutic agent to the ID compartment of the human subject's skin. Intradermal delivery of agents for the treatment, prevention, management of cancer, cancer metastasis or tumor growth results in a greater reduction of the tumor growth as compared to when the same agent is delivered by a route other than ID delivery (e.g., SC, IM, IV, epidermal). In some embodiments, intradermal delivery of agents for the treatment, prevention, management of cancer, cancer metastasis or tumor growth results in an increase in the median life span of the human subject as compared to when the agent is delivered by a route other than ID delivery.

Directly targeting the intradermal compartment as taught by the invention provides more rapid onset of effects of biologically active agents, including therapeutic agents, and higher bioavailability including, tissue bioavailability, relative to conventional modes of delivery of such agents, including subcutaneous delivery. The inventors have found that agents delivered in accordance with the methods of the invention can be rapidly absorbed and systemically distributed via controlled intradermal administration that selectively accesses the dermal vascular and lymphatic microcapillaries, thus the agents may exert their beneficial effects more rapidly than conventional modes of administration, such as subcutaneous administration.

As used herein, delivery to the intradermal compartment or intradermally delivered is intended to mean administration of a biologically active agent into the dermis in such a manner that the agent readily reaches the richly vascularized papillary dermis and is rapidly absorbed into the blood capillaries and/or lymphatic vessels to become systemically bioavailable. Such can result from placement of the agent in the upper region of the dermis, i.e., the papillary dermis or in the upper portion of the relatively less vascular reticular dermis such that the agent readily diffuses into the papillary dermis. The controlled delivery of a biologically active agent in this dermal compartment below the papillary dermis in the reticular dermis, but sufficiently above the interface between the dermis and the subcutaneous tissue, should enable an efficient (outward) migration of the agent to the (undisturbed) vascular and lymphatic microcapillary bed (in the papillary dermis), where it can be absorbed into systemic circulation via these microcapillaries without being sequestered in transit by any other cutaneous tissue compartment. In some embodiments, placement of a biologically active agent predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption of the agent. Although not intending to be bound by a particular mechanism of action, placement of the biologically active agent predominately at greater depths and/or into the lower portion of the reticular dermis may result in less effective uptake of the agent by the lymphatics as the agent will be slowly absorbed in the less vascular reticular dermis or in the subcutaneous region.

5.1 Methods of Intradermal Adminisration

The present invention encompasses methods for intradermal delivery of therapeutic agents or substances described and exemplified herein to the intradermal compartment of a subject's skin, preferably by selectively and specifically targeting the intradermal compartment without passing through it. In a most preferred embodiment, the intradermal compartment is targeted directly. Once a formulation containing the therapeutic agent to be delivered is prepared, the formulation is typically transferred to an injection device for intradermal compartment delivery, e.g., a syringe. Delivery of the formulations of the invention in accordance with the methods of the invention provides an improved therapeutic and clinical efficacy of the agent over conventional modes of delivery including IM and SC by specifically and selectively, preferably directly targeting the intradermal compartment. The intradermal delivery methods of the invention provide benefits and improvements over including but not limited to improved pharmacokinetics, rapid uptake into the local lymphatics, improved targeting to a particular tissue, and improved tissue bioavailability. The methods of the present invention result in an improved pharmacokinetics such as an improved absorption uptake within the intradermal compartment. The formulations of the invention may be delivered to the intradermal space as a bolus or by infusion.

The present invention encompasses methods for intradermal delivery of biologically active agents, particularly therapeutic agents such that the agent has a higher tissue bioavailability in a particular tissue as compared to when the agent is delivered by a route other than intradermal delivery such as SC delivery, intramuscular delivery, intravenous delivery, and epidermal delivery. In some embodiments, biologically active agents, particularly therapeutic agents delivered in accordance with the methods of the invention have a similar bioavailability, including tissue bioavailability as compared to when the agent is delivered intravenously.

The present invention encompasses methods for intradermal delivery of biologically active agents, particularly therapeutic agents such that the agent has a higher tissue bioavailability in a particular tissue as compared to when the agent is delivered to a deeper tissue compartment, e.g., SC. Preferably, the biologically active agent delivered in accordance with the methods of the invention is a therapeutic agent, administered for the treatment, prevention, delay of onset or the progression, or management of a disease including but not limited to cancer (e.g., lymphoma, leukemia, breast cancer, melanoma, lung cancer, renal cancer, and colorectal cancer), metastasis, tumor growth, or an infectious disease.

The present invention encompasses methods for intradermal delivery of biologically active agents, particularly therapeutic agents such that the agent has higher therapeutic efficacy as compared to when the agent is delivered to a deeper tissue compartment, e.g., SC. In some embodiments, biologically active agents, particularly therapeutic agents delivered in accordance with the methods of the invention have a similar therapeutic efficacy as compared to when the agent is delivered intravenously.

The invention encompasses methods of treating, preventing or management of a disease comprising administering at least one therapeutic agent at a pre-selected dose, wherein the pre-selected dose is reduced by at least half a fold, at least 5 fold, at least 10 fold compared to the dose of the agent that is conventionally delivered by other routes of delivery such as SC, IM, and IV.

In some embodiments, the invention encompasses methods of treating, preventing, or managing cancer, cancer metastasis or tumor growth in a human subject in need thereof, comprising delivering a therapeutic agent to the ID compartment of the human subject's skin. Intradermal delivery of agents for the treatment, prevention, management of cancer, cancer metastasis or tumor growth results in a greater reduction of the tumor growth as compared to when the same agent is delivered by a route other than ID delivery (e.g., SC, IM, IV, epidermal). In some embodiments, intradermal delivery of agents for the treatment, prevention, management of cancer, cancer metastasis or tumor growth results in an increase in the median life span of the human subject as compared to when the agent is delivered by a route other than ID delivery

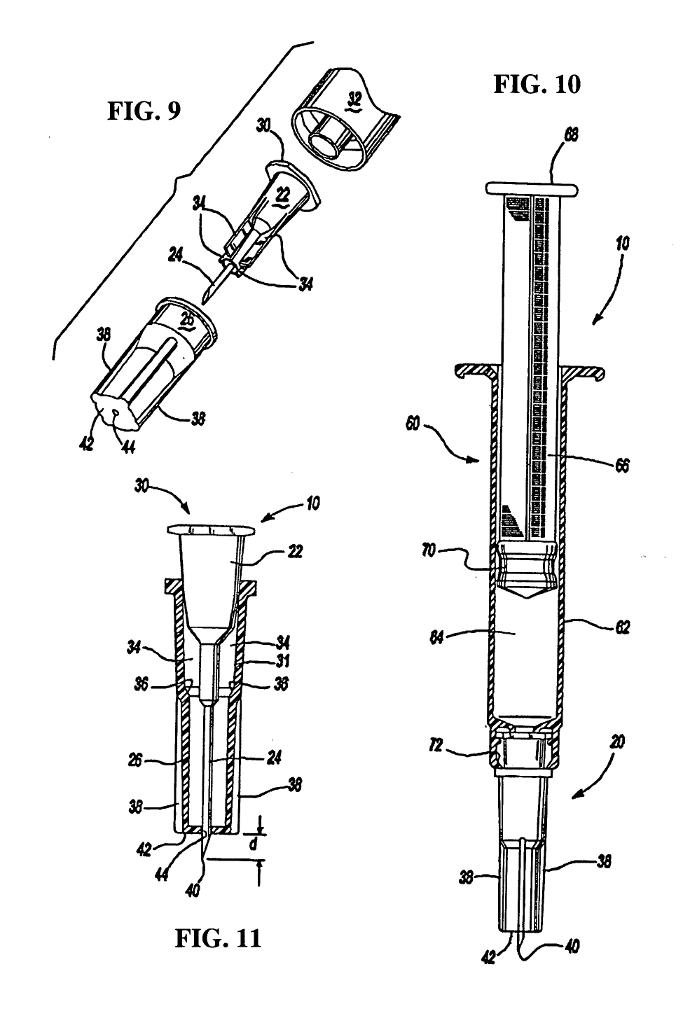

In accordance with the invention direct intradermal administration can be achieved using, for example, microneedle-based injection and infusion systems or any other means known to one skilled in the art to accurately target the intradermal compartment. Particular devices include those disclosed in WO 01/02178, published Jan. 10, 2002; and WO 02/02179, published Jan. 10, 2002, U.S. Pat. No. 6,494,865, issued Dec. 17, 2002 and U.S. Pat. No. 6,569,143 issued May 27, 2003 all of which are incorporated herein by reference in their entirety, as well as those exemplified in FIGS. 9-11. Micro-cannula- and microneedle-based methodology and devices are also described in U.S. application Ser. No. 09/606,909, filed Jun. 29, 2000, which is incorporated herein by reference in its entirety. Standard steel cannula can also be used for intra-dermal delivery using devices and methods as described in U.S. Ser. No. 417,671, filed Oct. 14, 1999, which is incorporated herein by reference in its entirety. These methods and devices include the delivery of agents through narrow gauge (30 G or narrower) “micro-cannula” with a limited depth of penetration (typically ranging from 10 μm to 2 mm), as defined by the total length of the cannula or the total length of the cannula that is exposed beyond a depth-limiting hub feature.

The subject of intradermal delivery of the present invention is a mammal, preferably, a human. The biologically active agents delivered in accordance with the methods of the invention may be delivered into the intradermal compartment by a needle or cannula, usually from about 300 μm to about 5 mm long. Preferably, the needle or cannula is about 300 μm to about 1 mm long, with the outlet inserted into the skin of the subject to a depth of 1 mm to 3 mm. Preferably, a small gauge needle or cannula, between 30 and 36 gauge, preferably 31-34 gauge is used. The outlet of the needle or cannula is preferably inserted to a depth of 0.3 mm (300 um) to 1.5 mm.

In accordance with the invention direct intradermal administration can be achieved using, for example, microneedle-based injection and infusion systems or any other means known to one skilled in the art to accurately target the intradermal compartment. Particular devices include those disclosed in WO 01/02178, published Jan. 10, 2002; and WO 02/02179, published Jan. 10, 2002, U.S. Pat. No. 6,494,865, issued Dec. 17, 2002 and U.S. Pat. No. 6,569,143 issued May 27, 2003 all of which are incorporated herein by reference in their entirety, as well as those exemplified in FIGS. 9-11. Micro-cannula- and microneedle-based methodology and devices are also described in U.S. application Ser. No. 09/606,909, filed Jun. 29, 2000, which is incorporated herein by reference in its entirety. Standard steel cannula can also be used for intra-dermal delivery using devices and methods as described in U.S. Ser. No. 417,671, filed Oct. 14, 1999, which is incorporated herein by reference in its entirety. These methods and devices include the delivery of agents through narrow gauge (30 G or narrower) “micro-cannula” with a limited depth of penetration (typically ranging from 10 μm to 2 mm), as defined by the total length of the cannula or the total length of the cannula that is exposed beyond a depth-limiting hub feature.

The intradermal methods of administration comprise microneedle-based injection and infusion systems or any other means to accurately target the intradermal space. The intradermal methods of administration encompass not only microdevice-based injection means, but other delivery methods such as needless or needle-free ballistic injection of fluids or powders into the intradermal space, Mantoux-type intradermal injection, enhanced iontophoresis through microdevices, and direct deposition of fluid, solids, or other dosing forms into the skin.

In a specific embodiment, the formulations of the invention are administered to an intradermal compartment of a subject's skin using an intradermal Mantoux type injection, see, e.g., Flynn et al, 1994, Chest 106: 1463-5, which is incorporated herein by reference in its entirety. In a specific embodiment, the formulation of the invention is delivered to the intradermal compartment of a subject's skin using the following exemplary method. The formulation is drawn up into a syringe, e.g., a 1 mL latex free syringe with a 20 gauge needle; after the syringe is loaded it is replaced with a 30 gauge needle for intradermal administration. The skin of the subject, e.g., mouse, is approached at the most shallow possible angle with the bevel of the needle pointing upwards, and the skin pulled tight. The injection volume is then pushed in slowly over 5-10 seconds forming the typical “bleb” and the needle is subsequently slowly removed. Preferably, only one injection site is used. More preferably, the injection volume is no more than 100 μL, due in part, to the fact that a larger injection volume may increase the spill over into the surrounding tissue space, e.g., the subcutaneous space.

The invention encompasses the use of conventional injection needles, catheters or microneedles of all known types, employed singularly or in multiple needle arrays. The terms “needle” and “needles” as used herein are intended to encompass all such needle-like structures. The term “microneedles” as used herein are intended to encompass structures smaller than about 30 gauge, typically about 31-50 gauge when such structures are cylindrical in nature. Non-cylindrical structures encompass by the term microneedles would therefore be of comparable diameter and include pyramidal, rectangular, octagonal, wedged, and other geometrical shapes.

The invention encompasses ballistic fluid injection devices, powder jet delivery devices, piezoelectric, electromotive, electromagnetic assisted delivery devices, gas-assisted delivery devices, which directly penetrate the skin to directly deliver the formulations of the invention to the targeted location within the dermal space.

The actual method by which the formulations of the invention are targeted to the intradermal space is not critical as long as it penetrates the skin of a subject to the desired targeted depth within the intradermal space without passing through it. The actual optimal penetration depth will vary depending on the thickness of the subject's skin. In most cases, skin is penetrated to a depth of about 0.5-2 mm. Regardless of the specific intradermal device and method of delivery, the methods of the invention preferably targets the formulations of the invention to a depth of at least at least 0.5 mm up to a depth of no more than 2.5 mm, more preferably no more than 2.0 mm, and most preferably no more than 1.7 mm. In some embodiments, the formulations are delivered at a targeted depth just under the stratum corneum and encompassing the epidermis and upper dermis, e.g., about 0.025 mm to about 2.5 mm. In order to target specific cells in the skin, the preferred target depth depends on the particular cell being targeted and the thickness of the skin of the particular subject. For example, to target the Langerhan's cells in the dermal space of human skin, delivery would need to encompass, at least, in part, the epidermal tissue depth typically ranging from about 0.025 mm to about 0.2 mm in humans.

The formulations delivered or administered in accordance with the invention include solutions thereof in pharmaceutically acceptable diluents or solvents, suspensions, gels, particulates such as micro- and nanoparticles either suspended or dispersed, as well as in-situ forming vehicles of same.

In other preferred embodiments, the invention encompass selecting an injection site on the skin of the subject, cleaning the injection site on the skin of the subject prior to expelling the biologically active agents, particularly therapeutic agents from the delivery device into the skin of the subject. In addition, the method comprises filling the delivery device with the biologically active agents, particularly therapeutic agents of the invention. Further, the method comprises pressing the skin engaging surface of the limiter portion against the skin of the subject and applying pressure, thereby stretching the skin of the subject, and withdrawing the needle cannula from the skin after injecting the agent. Still further, the step of inserting the forward tip into the skin is further defined by inserting the forward tip into the skin to a depth of from approximately 1.0 mm to approximately 2.0 mm, and most preferably into the skin to a depth of 1.5 mm+0.2 to 0.3 mm.

In a preferred embodiment, the step of inserting the forward tip into the skin of the animal is further defined by inserting the forward tip into the skin at an angle being generally perpendicular to the skin within about fifteen degrees, with the angle most preferably being generally ninety degrees to the skin, within about five degrees, and the fixed angle of orientation relative to the skin engaging surface is further defined as being generally perpendicular. In the preferred embodiment, the limiter surrounds the needle cannula, having a generally planar flat skin engaging surface. Also, the delivery device comprises a syringe having a barrel and a plunger received within the barrel and the plunger being depressable to expel the agent from the delivery device through the forward tip of the needle cannula.

In a preferred embodiment, expelling the biologically active agents, particularly therapeutic agents, from the delivery device is further defined by grasping the hypodermic needle with a first hand and depressing the plunger with an index finger of a second hand and expelling the agent from the delivery device by grasping the hypodermic needle with a first hand and depressing the plunger on the hypodermic needle with a thumb of a second hand, with the step of inserting the forward tip into the skin of the animal further defined by pressing the skin of the animal with the limiter. In addition, the method may further comprise the step of attaching a needle assembly to a tip of the barrel of the syringe with the needle assembly including the needle cannula and the limiter, and may comprise the step of exposing the tip of the barrel before attaching the needle assembly thereto by removing a cap from the tip of the barrel. Alternatively, the step of inserting the forward tip of the needle into the skin of the subject may be further defined by simultaneously grasping the hypodermic needle with a first hand and pressing the limiter against the skin of the animal thereby stretching the skin of the animal, and expelling the agent by depressing the plunger with an index finger of the first hand or expelling the agent by depressing the plunger with a thumb of the first hand. The method further encompasses withdrawing the forward tip of the needle cannula from the skin of the subject after the agent has been injected into the skin of the subject. Still further, the method encompasses inserting the forward tip into the skin preferably to a depth of from approximately 1.0 mm to approximately 2.0 mm, and most preferably to a depth of 1.5 mm+0.2 to 0.3 mm.

It has been found that certain features of the intradermal administration methods provide clinically useful PK/PD and dose accuracy. For example, it has been found that placement of the needle outlet within the skin significantly affects PK/PD parameters. The outlet of a conventional or standard gauge needle with a bevel has a relatively large exposed height (the vertical rise of the outlet). Although the needle tip may be placed at the desired depth within the intradermal compartment, the large exposed height of the needle outlet causes the delivered agent to be deposited at a much shallower depth nearer to the skin surface. As a result, the agent tends to effuse out of the skin due to backpressure exerted by the skin itself and to pressure built up from accumulating fluid from the injection or infusion and to leak into the lower pressure regions of the skin, such as the subcutaneous tissue. That is, at a greater depth a needle outlet with a greater exposed height will still seal efficiently where as an outlet with the same exposed height will not seal efficiently when placed in a shallower depth within the intradermal compartment. Typically, the exposed height of the needle outlet will be from 0 to about 1 mm. A needle outlet with an exposed height of 0 mm has no bevel and is at the tip of the needle. In this case, the depth of the outlet is the same as the depth of penetration of the needle. A needle outlet that is either formed by a bevel or by an opening through the side of the needle has a measurable exposed height. It is understood that a single needle may have more than one opening or outlets suitable for delivery of agents to the dermal compartment.

It has also been found that by controlling the pressure of injection or infusion the high backpressure exerted during ID administration can be overcome. By placing a constant pressure directly on the liquid interface a more constant delivery rate can be achieved, which may optimize absorption and obtain the improved pharmacokinetics. Delivery rate and volume can also be controlled to prevent the formation of wheals at the site of delivery and to prevent backpressure from pushing the dermal-access means out of the skin and/or into the subcutaneous region. The appropriate delivery rates and volumes to obtain these effects may be determined experimentally using only ordinary skill. Increased spacing between multiple needles allows broader fluid distribution and increased rates of delivery or larger fluid volumes.

The administration methods useful for carrying out the invention include both bolus and infusion delivery of the biologically active agents to humans or animals subjects. A bolus dose is a single dose delivered in a single volume unit over a relatively brief period of time, typically less than about 10 minutes. Infusion administration comprises administering a fluid at a selected rate that may be constant or variable, over a relatively more extended time period, typically greater than about 10 minutes. To deliver an agent, the dermal-access means is placed adjacent to the skin of a subject providing directly targeted access within the intradermal compartment and the agent or agents are delivered or administered into the intradermal compartment where they can act locally or be absorbed by the bloodstream and be distributed systematically. The dermal-access means may be connected to a reservoir containing the agent or agents to be delivered.

Delivery from the reservoir into the intradermal compartment may occur either passively, without application of the external pressure or other driving means to the agent or agents to be delivered, and/or actively, with the application of pressure or other driving means. Examples of preferred pressure generating means include pumps, syringes, pens, elastomer membranes, gas pressure, piezoelectric, electromotive, electromagnetic or osmotic pumping, or Belleville springs or washers or combinations thereof. If desired, the rate of delivery of the agent may be variably controlled by the pressure-generating means.

In some embodiments, the invention encompasses methods for controlling the pharmacokinetics of administered biologically active agents by combining the advantages of delivery to two or more compartments or depths within skin. In particular, the invention provides a method for delivering a biologically active agent, particularly a therapeutic agent as described herein to the shallow SC and ID compartments to achieve a hybrid pK profile that has a portion similar to that achieved by ID delivery and another portion similar to that achieved by SC delivery. This provides rapid and high peak onset levels of the biologically active agent, particularly a therapeutic agent as well as a lower prolonged circulating level of the agent. Such methods are disclosed in U.S. application Ser. No. 10/429,973, filed May 6, 2003 which is incorporated herein by reference in its entirety. In some embodiments, the biologically active agent, particularly a therapeutic agent is delivered to a site or, sites that include two or more compartments. In other embodiments, biologically active agent, particularly a therapeutic agent is delivered to multiple sites that each include one or more compartments.

The methods of the invention encompass controlled delivery of the biologically active agent, particularly a therapeutic agent using algorithms having logic components that include physiologic models, rules based models or moving average methods, therapy pharmacokinetic models, monitoring signal processing algorithms, predictive control models, or combinations thereof.

The methods of the invention encompass a method for combinations of shallow SC and ID delivery to achieve improved PK outcomes. These outcomes are not achievable using solely one delivery compartment or another. Multiple site deposition via proper device configuration and/or dosing method may obtain unique and beneficial results. The underlying technical principle is that the PK outcome of microneedle delivery is specific to the deposition depth and patterning of the administered fluid, that such deposition can be controlled mechanically via device design and engineering or by technique such as fluid overloading of the ID compartment.

In addition, the invention includes needles (micro or otherwise) for subcutaneous injection having a length less than 5 mm length. Shallow SC delivery to a depth of about 3 mm yields almost identical PK to deep SC using traditional techniques. The utility of shallow SC delivery alone to yield more controlled profiles has never been exploited. In fact, previously depths of less than 5mm have been considered to not be within the SC compartment.

Mixed delivery either by device design or technique results in biphasic or mixed kinetic profiling. Minor differences in device length (1 mm vs. 2 mm vs. 3 mm) 30 yield dramatic differences in PK outcomes. SC-like profiles can be obtained with needle lengths often assumed to locate the end of the needle within the ID compartment. Shallow SC delivery is more consistent and uniform in PK outcomes than standard SC delivery. The limits of the targeted tissue depth are controlled inter alia by the depth to which the needle or cannula outlet is inserted, the exposed height (vertical rise) of the outlet, the volume administered, and the rate of administration. Suitable parameters can be determined by persons of skill in the art without undue experimentation.

5.1.1 Devices for Intradermal Adminisration

The biologically active agents, including the therapeutic agents of the invention are administered using any of the devices and methods known in the art or disclosed in WO 01/02178, published Jan. 10, 2002; and WO 02/02179, published Jan. 10, 2002, U.S. Pat. No. 6,494,865, issued Dec. 17, 2002 and U.S. Pat. No. 6,569,143 issued May 27, 2003 all of which are incorporated herein by reference in their entirety.

Preferably the devices for intradermal administration in accordance with the methods of the invention have structural means for controlling skin penetration to the desired depth within the intradermal space. This is most typically accomplished by means of a widened area or hub associated with the shaft of the dermal-access means that may take the form of a backing structure or platform to which the needles are attached. The length of microneedles as dermal-access means are easily varied during the fabrication process and are routinely produced in less than 2 mm length. Microneedles are also a very sharp and of a very small gauge, to further reduce pain and other sensation during the injection or infusion. They may be used in the invention as individual single-lumen microneedles or multiple microneedles may be assembled or fabricated in linear arrays or two-dimensional arrays as to increase the rate of delivery-or the amount of substance delivered in a given period of time. The needle may eject its substance from the end, the side or both. Microneedles may be incorporated into a variety of devices such as holders and housings that may also serve to limit the depth of penetration. The dermal-access means of the invention may also incorporate reservoirs to contain the substance prior to delivery or pumps or other means for delivering the drug or other substance under pressure. Alternatively, the device housing the dermal-access means may be linked externally to such additional components.

The intradermal methods of administration comprise microneedle-based injection and infusion systems or any other means to accurately target the intradermal space. The intradermal methods of administration encompass not only microdevice-based injection means, but other delivery methods such as needle-less or needle-free ballistic injection of fluids or powders into the intradermal space, enhanced ionotophoresis through microdevices, and direct deposition of fluid, solids, or other dosing forms into the skin.

In some embodiments, the present invention provides a delivery device including a needle assembly for use in making intradermal injections. The needle assembly has an adapter that is attachable to prefillable containers such as syringes and the like. The needle assembly is supported by the adapter and has a hollow body with a forward end extending away from the adapter. A limiter surrounds the needle and extends away from the adapter toward the forward end of the needle. The limiter has a skin engaging surface that is adapted to be received against the skin of an animal such as a human. The needle forward end extends away from the skin engaging surface a selected distance such that the limiter limits the amount or depth that the needle is able to penetrate through the skin of an animal

In a specific embodiment, the hypodermic needle assembly for use in the methods of the invention comprises the elements necessary to perform the present invention directed to an improved method delivering biologically active agents, including the therapeutic agents into the skin of a subject's skin, preferably a human subject's skin, comprising the steps of providing a delivery device including a needle cannula having a forward needle tip and the needle cannula being in fluid communication with an agent contained in the delivery device and including a limiter portion surrounding the needle cannula and the limiter portion including a skin engaging surface, with the needle tip of the needle cannula extending from the limiter portion beyond the skin engaging surface a distance equal to approximately 0.5 mm to approximately 3.0 mm and the needle cannula having a fixed angle of orientation relative to a plane of the skin engaging surface of the limiter portion, inserting the needle tip into the skin of an animal and engaging the surface of the skin with the skin engaging surface of the limiter portion, such that the skin engaging surface of the limiter portion limits penetration of the needle cannula tip into the dermis layer of the skin of the animal, and expelling the substance from the drug delivery device through the needle cannula tip into the skin of the animal.

In a specific embodiment, the invention encompasses a drug delivery device as disclosed in FIG. 9-FIG. 10 illustrate an example of a drug delivery device which can be used to practice the methods of the present invention for making intradermal injections. The device 10 illustrated in FIGS. 9-10. includes a needle assembly 20 which can be attached to a syringe barrel 60. Other forms of delivery devices may be used including pens of the types disclosed in U.S. Pat. No. 5,279,586, U.S. patent application Ser. No. 09/027,607 and PCT Application No. WO 00/09135, the disclosure of which are hereby incorporated by reference in their entirety.

The needle assembly 20 includes a hub 22 that supports a needle cannula 24. The limiter 26 receives at least a portion of the hub 22 so that the limiter 26 generally surrounds the needle cannula 24 as best seen in FIG. 9.

One end 30 of the hub 22 is able to be secured to a receiver 32 of a syringe. A variety of syringe types for containing the substance to be intradermally delivered according to the present invention can be used with a needle assembly designed, with several examples being given below. The opposite end of the hub 22 preferably includes extensions 34 that are nestingly received against abutment surfaces 36 within the limiter 26. A plurality of ribs 38 preferably are provided on the limiter 26 to provide structural integrity and to facilitate handling the needle assembly 20.

By appropriately designing the size of the components, a distance “d” between a forward end or tip 40 of the needle 24 and a skin engaging surface 42 on the limiter 26 can be tightly controlled. The distance “d” preferably is in a range from approximately 0.5 mm to approximately 3.0 mm, and most preferably around 1.5 mm±0.2 mm to 0.3 mm. When the forward end 40 of the needle cannula 24 extends beyond the skin engaging surface 42 a distance within that range, an intradermal injection is ensured because the needle is unable to penetrate any further than the typical dermis layer of an animal. Typically, the outer skin layer, epidermis, has a thickness between 50-200 microns, and the dermis, the inner and thicker layer of the skin, has a thickness between 1.5-3.5 mm. Below the dermis layer is subcutaneous tissue (also sometimes referred to as the hypodermis layer) and muscle tissue, in that order.

As can be best seen in FIG. 9, the limiter includes an opening 44 through which the forward end 40 of the needle cannula 24 protrudes. The dimensional relationship between the opening 44 and the forward end 40 can be controlled depending on the requirements of a particular situation. In the illustrated embodiment, the skin engaging surface 42 is generally planar or flat and continuous to provide a stable placement of the needle assembly 20 against an animal's skin. Although not specifically illustrated, it may be advantageous to have the generally planar skin engaging surface 42 include either raised portions in the form of ribs or recessed portions in the form of grooves in order to enhance stability or facilitate attachment of a needle shield to the needle tip 40. Additionally, the ribs 38 along the sides of the limiter 26 may be extended beyond the plane of the skin engaging surface 42.

Regardless of the shape or contour of the skin engaging surface 42, the preferred embodiment includes enough generally planar or flat surface area that contacts the skin to facilitate stabilizing the injector relative to the subject's skin. In the most preferred arrangement, the skin engaging surface 42 facilitates maintaining the injector in a generally perpendicular orientation relative to the skin surface and facilitates the application of pressure against the skin during injection. Thus, in the preferred embodiment, the limiter has dimension or outside diameter of at least 5 mm. The major dimension will depend upon the application and packaging limitations, but a convenient diameter is less than 15 mm or more preferably 11-12 mm.

It is important to note that although FIGS. 9 and 10 illustrate a two-piece assembly where the hub 22 is made separate from the limiter 26, a device for use in connection with the invention is not limited to such an arrangement. Forming the hub 22 and limiter 26 integrally from a single piece of plastic material is an alternative to the example shown in FIGS. 9 and 10. Additionally, it is possible to adhesively or otherwise secure the hub 22 to the limiter 26 in the position illustrated in FIGS. 10 so that the needle assembly 20 becomes a single piece unit upon assembly.

Having a hub 22 and limiter 26 provides the advantage of making an intradermal needle practical to manufacture. The preferred needle size is a small Gauge hypodermic needle, commonly known as a 30 Gauge or 31 Gauge needle. Having such a small diameter needle presents a challenge to make a needle short enough to prevent undue penetration beyond the dermis layer of an animal. The limiter 26 and the hub 22 facilitate utilizing a needle 24 that has an overall length that is much greater than the effective length of the needle, which penetrates the individual's tissue during an injection. With a needle assembly designed in accordance herewith, manufacturing is enhanced because larger length needles can be handled during the manufacturing and assembly processes while still obtaining the advantages of having a short needle for purposes of completing an intradermal injection.

FIG. 11 illustrates the needle assembly 20 secured to a drug container such as a syringe 60 to form the device 10. A generally cylindrical syringe body 62 can be made of plastic or glass as is known in the art. The syringe body 62 provides a reservoir 64 for containing the substance to be administered during an injection. A plunger rod 66 has a manual activation flange 68 at one end with a stopper 70 at an opposite end as known in the art. Manual movement of the plunger rod 66 through the reservoir 64 forces the substance within the reservoir 64 to be expelled out of the end 40 of the needle as desired.

The hub 22 can be secured to the syringe body 62 in a variety of known manners. In one example, an interference fit is provided between the interior of the hub 22 and the exterior of the outlet port portion 72 of the syringe body 62. In another example, a conventional Luer fit arrangement is provided to secure the hub 22 on the end of the syringe 60. As can be appreciated from FIGS. 9-11, such needle assembly designed is readily adaptable to a wide variety of conventional syringe styles.

This invention provides an intradermal needle injector that is adaptable to be used with a variety of syringe types. Therefore, this invention provides the significant advantage of facilitating manufacture and assembly of intradermal needles on a mass production scale in an economical fashion.

Prior to inserting the needle cannula 24, an injection site upon the skin of the animal is selected and cleaned. Subsequent to selecting and cleaning the site, the forward end 40 of the needle cannula 24 is inserted into the skin of the animal at an angle of generally 90 degrees until the skin engaging surface 42 contacts the skin. The skin engaging surface 42 prevents the needle cannula 42 from passing through the dermis layer of the skin and injecting the substance into the subcutaneous layer.

While the needle cannula 42 is inserted into the skin, the substance is intradermally injected. The substance may be prefilled into the syringe 60, either substantially before and stored therein just prior to making the injection. Several variations of the method of performing the injection may be utilized depending upon individual preferences and syringe type. In any event, the penetration of the needle cannula 42 is most preferably no more than about 1.5 mm because the skin engaging surface 42 prevents any further penetration.

Also, during the administration of an intradermal injection, the forward end 40 of the needle cannula 42 is embedded in the dermis layer of the skin which results in a reasonable amount of back pressure during the injection of the substance. This back pressure could be on the order of 76 psi. In order to reach this pressure with a minimal amount of force having to be applied by the user to the plunger rod 66 of the syringe, a syringe barrel 60 with a small inside diameter is preferred such as 0.183″ (4.65 mm) or less. The method of this invention thus includes selecting a syringe for injection having an inside diameter of sufficient width to generate a force sufficient to overcome the back pressure of the dermis layer when the substance is expelled from the syringe to make the injection.

In addition, since intradermal injections are typically carried out with small volumes of the substance to be injected, i.e., on the order of no more than 0.5 ml, and preferably around 0.1 ml, a syringe barrel 60 with a small inside diameter is preferred to minimize dead space which could result in wasted substance captured between the stopper 70 and the shoulder of the syringe after the injection is completed. Also, because of the small volumes of substance, on the order of 0.1 ml, a syringe barrel with a small inside diameter is preferred to minimize air head space between the level of the substance and the stopper 70 during process of inserting the stopper. Further, the small inside diameter enhances the ability to inspect and visualize the volume of the substance within the barrel of the syringe.

5.2 Treating Disorders in Accordance with the Methods

The present invention encompasses administering one or more of the therapeutic agents to an animal, preferably a mammal, and most preferably a human, for preventing, treating, or ameliorating one or more symptoms associated with a disease, disorder, or infection, by delivering the agent to the ID compartment of the subject's skin. The methods of the invention are particularly useful for the treatment or prevention of a disease or disorder of the lymphatic system, primary or metastatic neoplastic disease (i.e., cancer), and infectious diseases. Therapeutic agents may be provided in pharmaceutically acceptable compositions or formulations as known in the art or as described herein.

The invention encompasses methods of treating, preventing or managing a disease or disorder in a subject in need thereof, said method comprising administering to said subject a therapeutically effective amount or prophylactically effective amount of one or more therapeutic agents to the intradermal compartment of the subject's skin.

The present invention provides a method of treating or preventing a disease in a subject by delivering a therapeutic agent to the intradermal compartment in a subject such that the therapeutic agent is more effective as compared to conventional delivery routes, e.g., IM, IV or SC.

In some embodiments, the invention also encompasses methods for treating or preventing an infectious disease in a subject comprising administering a therapeutically or prophylatically effective amount of one or more therapeutic agents that bind an infectious agent or cellular receptor therefor. Infectious diseases that can be treated or prevented by the molecules of the invention are caused by infectious agents including but not limited to viruses, bacteria, fungi, protozae, and viruses.

Intradermal delivery of biologically active agents in accordance with the present invention provides among other benefits, rapid uptake into local lymphatics, improved targeting and deposition of the delivered agent to a particular tissue, and improved systemic and tissue bioavailability. Such benefits are especially useful for the delivery of therapeutic agents, such as antineoplastic agents, antibodies and antibiotics. Intradermal delivery of agents in accordance with the methods of the invention deposits the agent into the intradermal and lymphatic compartments and deeper tissues, resulting in rapid and biologically significant concentrations of the agents in these compartments and tissues.

By direct lymphatic targeting of various therapeutic agents drug entities using intradermal delivery beneficial therapeutic outcomes are realized, including dose sparing, increased drug efficacy, reduced side effects, reduced metastatic potential, and prolonged survival. The present invention provides improved methods for treating diseases in that the delivery methods of the invention allow for both systemic and localized deposition of therapeutic agents. As a result, the present invention provides improved methods for treatment of a diseases such as cancer, by improving sensitivity, the amount of the agent deposited, tissue bioavailability, faster onset and clearance of the delivered therapeutic agent. The invention provides a method for administration of at least one therapeutic agent for the treatment of a disease, particularly cancer, comprising delivering the agent into the intradermal compartment of a subject's skin at a controlled rate, volume and pressure so that the agent is deposited into the ID compartment and taken up by the lymphatic vasculature.

The present invention also provides improved methods for treatment of a diseases that are localized to particular tissues and organs of the body, such as infection of those tissues and organs, e.g., respiratory infection, by improving sensitivity, the amount of the agent deposited, tissue bioavailability, faster onset and clearance of the delivered therapeutic agent. The invention provides a method for administration of at least one therapeutic agent for the treatment of a disease, particularly infection, comprising delivering the agent into the intradermal compartment of a subject's skin at a controlled rate, volume and pressure so that the agent is deposited into the intradermal compartment and taken up by the lymphatic vasculature.

In a particular embodiment, the present invention provides an improved method for delivery of antineoplastic agents, chemotherapeutic agents, antibodies, anti-angiogenesis agents, anti-inflammatory agents, immunotherapeutic agents, etc. with improved clinical utility and therapeutic efficacy. Conventional therapy of primary cancer lesions is routinely accomplished via either surgical resection of the primary mass, localized radiation therapy to kill the tumor tissue, or administration of systemic antineoplastic drugs to kill the tumor. The first two methods have the advantage of being more local in scope and when available for treatment can potentially minimize the damage to non-target organs. Conversely, because this treatment is localized, the potential for affecting metatstaic cells which have been shed from the primary tumor and localized in other tissues is limited. Systemic antineoplastic therapy has the potential of affecting disseminated tumor cells as well as the primary tumor, but this systemic delivery increases the potential for damage of healthy organs, tissues, and systems.

Intradermal delivery of therapeutic agents, such as antibodies and antineoplastic agents, in accordance with the present invention can directly access both the venous and lymphatic networks of the dermis and provide unique systemic phamacokinetic outcomes. By accessing these networks in the vicinity of the target, such as the tumor, therapeutic advantages may be achieved. One, localized effects will be enhanced relative to systemic toxicity of the adverse events, since the intradermally delivered antineoplastic agent is physically placed in the vicinity of the tumor, leading to reduced dosages and reduced side effects. Likewise, since tumors principally use systemic vasculature for metastatic trafficking, the intradermally delivered antineoplastic agents can target these shed cells, and reduce the potential for metasteses. The intradermally administered antineoplastic agents may also access the immune cells mediated by the lymphatic network affecting immunological cell maturation and trafficking of anti-cancer cells (T, B, NK, macrophages, etc) to tumor sites. Further, intradermally delivered antineoplastic agents will result in systemic distribution providing the added benefit of reaching extensively disseminated sites and organs, in a manner similar to many current therapies. The present invention provides an improved method of enhancing the bioavailability of a biologically active agent to a particular tissue, including but not limited to lymphatic, mucosal, lung, spleen, thymus or colon tissue. Intradermal delivery of biologically active agents in accordance with the present invention provides among other benefits, rapid uptake into local lymphatics, improved targeting and deposition of the delivered agent to a particular tissue, and improved systemic and tissue bioavailability. Such benefits are especially useful for the delivery of therapeutics, such as antineoplastic agents, antibodies and antibiotics. Intradermal delivery of agents in accordance with the methods of the invention deposits the agent into the intradermal and lymphatic compartments and deeper tissues, resulting in rapid and biologically significant concentrations of the agents in these compartments and tissues.

Intradermal therapy may have greater benefits for certain cancer types. Peripheral cancers or ones that are highly localized, resident in, or associated with the vasculatures of interest may enjoy the greatest benefits of this therapy type. Specific cancers of exceptional benefit include, but are not limited to, melanomas and other cancers of the skin (sarcomas, etc), lymphomas or other cancers of the lymphoid tissue, breast cancers or other cancers of the peripheral soft tissues or subcutaneous spaces, splenic cancers which are in communication with the lymphatics, and leukemias or other cancers of the vasculature. The effects on cancer localized deep within the body, or within organs, or within privileged biological spaces with minimal drug transport mechanisms (brain, spine, etc) may also have benefit. In particular, intradermally delivered antineoplastic agents in accordance with the present invention, may be used to treat lung cancers as well.

The enhanced effects of intradermally delivered antineoplastic agents may differ for agents having different biological mechanisms of effects. Cytotoxic drugs may show greater initial tumor localization and killing prior to systemic distribution, thereby enhancing killing of the target tissues and minimizing side effects. Cytotoxic drugs however would necessitate a formulation which protects the tissue at the administration site from immediate death or necrosis upon administration. Encapsulation of the cytotoxic agents in a short lived liposome, particles or other carrier which shield the surrounding tissue may enhance the benefits of intradermal administration. Drugs which initiate a host response toward the tumor or stimulate a suppressed response would be of potentially greater benefit again owing to localization of the response in the tumor vicinity. Drugs which affect the immunological systems, chemokines, cytokines, and other immunopotentiators (the examples with IL-12 are a prime example) would likely have exceptional benefit via this delivery route. The use of drugs which incorporate both a chemical targeting of the tumor (e.g., tumor specific antibodies, receptors which target tumor surface markers, and markers which bind to tumor specific receptors) would potentially enjoy the highest benefit since they both physically and chemically target the tumor cells. Drugs of this class include, but are not limited to, therapeutic antibodies, particles carrying cytotoxic or other drugs that carry a tumor specific targeting marker, or other agents which bind to the tumor to attack by inherent biological mechanisms (e.g., a cellular immunological response, or a complement mediated anti-tumor response).

The methods of the invention also include administering the antineoplastic agents with tumor cells, thus providing a vaccine effect against future tumor challenges.

The present invention provides improved methods for treatment of a disease, e.g., cancer, by improving the amount of the agent deposited, tissue bioavailability, faster onset and clearance of the delivered therapeutic agent. The invention provides a method for administration of at least one therapeutic agent for the treatment of a disease, particularly cancer, comprising delivering the agent into the ID compartment of a subject's skin at a controlled rate, volume and pressure so that the agent is deposited into the ID compartment and taken up by the lymphatic vasculature.

Cancers and related disorders that can be treated or prevented by methods and compositions of the present invention include, but are not limited to, the following: Leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including but not limited to, ocular melanoma such as iris melanoma, choroidal melanoma, and cilliary body melanoma, and retinoblastoma; vaginal cancers, including but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including but not limited to, squamous cell carcinoma, and adenocarcinoma; uterine cancers including but not limited to, endometrial carcinoma and uterine sarcoma; ovarian cancers including but not limited to, ovarian epithelial carcinoma, borderline tumor, germ cell tumor, and stromal tumor; esophageal cancers including but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including but not limited to hepatocellular carcinoma and hepatoblastoma, gallbladder cancers including but not limited to, adenocarcinoma; cholangiocarcinomas including but not limited to, pappillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including but not limited to, adenocarcinoma, leiomyosarcoma, and rhabdomyosarcoma; penal cancers; oral cancers including but not limited to, squamous cell carcinoma; basal cancers; salivary gland cancers including but not limited to, adenocarcinoma, mucoepidermoid carcinoma, and adenoidcystic carcinoma; pharynx cancers including but not limited to, squamous cell cancer, and verrucous; skin cancers including but not limited to, basal cell carcinoma, squamous cell carcinoma and melanoma, superficial spreading melanoma, nodular melanoma, lentigo malignant melanoma, acral lentiginous melanoma; kidney cancers including but not limited to, renal cell cancer, adenocarcinoma, hypemephroma, fibrosarcoma, transitional cell cancer (renal pelvis and/or uterer); Wilms' tumor; bladder cancers including but not limited to, transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J.B. Lippincott Co., Philadelphia and Murphy et al., 1997, Infonned Decisions: The Complete Book of Cancer Diagnosis, Treatment, and Recovery, Viking Penguin, Penguin Books U.S.A., Inc., United States of America).

Accordingly, the methods and compositions of the invention are also useful in the treatment or prevention of a variety of cancers or other abnormal proliferative diseases, including (but not limited to) the following: carcinoma, including that of the bladder, breast, colon, kidney, liver, lung, ovary, pancreas, stomach, prostate, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions of the invention in the ovary, bladder, breast, colon, lung, skin, pancreas, or uterus. In other specific embodiments, sarcoma, melanoma, or leukemia is treated or prevented by the methods and compositions of the invention.

The invention also encompasses methods for treating or preventing an infectious disease in a subject comprising administering a therapeutically or prophylatically effective amount of one or more agents to the ID of the subject's skin. Infectious diseases that can be treated or prevented by the molecules of the invention are caused by infectious agents including but not limited to viruses, bacteria, fungi, protozae, and viruses.

Viral diseases that can be treated or prevented using the methods of the invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, small pox, Epstein Barr virus, human immunodeficiency virus type I (HIV-I), human immunodeficiency virus type II (HIV-II), and agents of viral diseases such as viral miningitis, encephalitis, dengue or small pox

In some embodiments, the invention encompasses a method of treating or preventing a respiratory disease or disorder, particularly respiratory infections (viral and bacterial), comprising administering an effective amount of a therapeutic agent to the intradermal compartment of a subject in need of treatment. The invention encompasses methods of treating or preventing Respiratory infections of the upper respiratory tract (e.g., nose, ears, sinuses, and throat) and lower respiratory tract (e.g., trachea, bronchial tubes, and lungs). Examples of viruses that cause upper respiratory tract infections include rhinoviruses and influenza viruses A and B. Examples of lower respiratory viral infections are parainfluenza virus infections (“PIV”), respiratory syncytial virus (“RSV”), and bronchiolitis. Examples of bacteria that cause lower respiratory tract infections include Streptococcus pneumoniae that causes pneumonococcal pneumonia and Mycobacterium tuberculosis that causes tuberculosis. Respiratory infections caused by fungi include systemic candidiasis, blastomycosis crytococcosis, coccidioidomycosis, and aspergillosis. Respiratory infections may be primary or secondary infections.

The invention provides methods of preventing, managing, treating, or ameliorating a respiratory disease or disorder resulting from or associated with any viral infection, said methods comprising administering an effective amount of one or more therapeutic agents in accordance with the methods of the inventions. Examples of viruses which cause viral infections include, but are not limited to, retroviruses (e.g., human T-cell lymphotrophic virus (HTLV) types I and II and human immunodeficiency virus (HIV)), herpes viruses (e.g., herpes simplex virus (HSV) types I and II, Epstein-Barr virus, HHV6-HHV8, and cytomegalovirus), arenavirues (e.g., lassa fever virus), paramyxoviruses (e.g., morbillivirus virus, human respiratory syncytial virus, mumps, hMPV, and pneumovirus), adenoviruses, bunyaviruses (e.g., hantavirus), cornaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., influenza viruses A, B and C and PIV), papovaviruses (e.g., papillomavirues), picornaviruses (e.g., rhinoviruses, enteroviruses and hepatitis A viruses), poxviruses, reoviruses (e.g., rotavirues), togaviruses (e.g., rubella virus), and rhabdoviruses (e.g., rabies virus). Biological responses to a viral infection include, but not limited to, elevated levels of antibodies, increased proliferation and/or infiltration of T cells, increased proliferation and/or infiltration of B cells, epithelial hyperplasia, and mucin production. The invention also provides methods of preventing, treating, managing, or ameliorating viral respiratory infections, such as the common cold, viral pharyngitis, viral laryngitis, viral croup, viral bronchitis, influenza, parainfluenza viral diseases (“PIV”) (e.g., croup, bronchiolitis, bronchitis, pneumonia), and respiratory syncytial virus (“RSV”), metapneumavirus, and adenovirus diseases (e.g., febrile respiratory disease, croup, bronchitis, pneumonia), said method comprising administering an effective amount of one or more therapeutic agents.

In preferred embodiments, the invention encompasses methods of treating a disease or disorder, particularly a respiratory disease splitting the standard dose of a therapeutic agent for the disease between intranasal (IN) and systemic delivery. Persons of skill in the art can determine through routine experimentation the optimal ratio for a split dose. In the case of IN/IM delivery a ratio between 5/95 and 30/70 is preferred. The inventors have found that this leads to an immediate level of the therapeutic agent, e.g., antibody in the tissues, e.g., lungs consistent with concentrations that neutralize high titers of virus in vitro. Further, splitting the dose did not lead to a detectable loss in the prophylactic concentration that is necessary to maintain for weeks after administration.

Bacterial diseases that can be treated or prevented using the methods of the invention in conjunction with the methods of the present invention, that are caused by bacteria include, but are not limited to, mycobacteria rickettsia, mycoplasma, neisseria, S. pneumonia, Borrelia burgdorferi (Lyme disease), Bacillus antracis (anthrax), tetanus, streptococcus, staphylococcus, mycobacterium, tetanus, pertissus, cholera, plague, diptheria, chlamydia, S. aureus and legionella.

Protozoal diseases that can be treated or prevented using the methods of the invention in conjunction with the methods of the present invention, that are caused by protozoa include, but are not limited to, leishmania, kokzidioa, trypanosoma or malaria.

Parasitic diseases that can be treated or prevented using the methods of the invention in conjunction with the methods of the present invention, that are caused by parasites include, but are not limited to, chlamydia and rickettsia.

5.3 Agents to be Administered in Accordance with the Invention

The present invention encompasses biologically active agents, particularly therapeutic agents, for treatment, prevention, or mangement of a disease or disorder. Examples of biologically active agents that can be used in the methods of the instant invention include without limitation, immunoglobulins (e.g., Multi-specific Igs, Single chain Igs, Ig fragments), Proteins, Peptides (e.g., Peptide receptors, PNAs, Selectins, binding proteins (maltose binding protein, glucose binding protein)), Nucleotides, Nucleic Acids (e.g., PNAS, RNAs, modified RNA/DNA, aptamers), Receptors (e.g., Acetylcholine receptor), Enzymes (e.g., Glucose Oxidase, HIV Protease and reverse transcriptase), Carbohydrates (e.g, NCAMs, Sialic acids), Cells (e.g., Insulin & Glucose responsive cells), bacteriophags (e.g., filamentous phage), viruses (e.g., HIV), Chemospecific agents (e.g., Cyptands, Crown ethers, Boronates).

The present invention provides methods for administering antineoplastic agents. Such antineoplastic agents include a variety of agents including cytokines, angiogenesis inhibitors, classic anticancer agents and therapeutic antibodies. Cytokines immunomodulating agents and hormones that may be used in accordance with the invention include, but are not limited to interferons, interleukins (IL-1, -2, -4, -6, -8, -12) and cellular growth factors.

Angiogenesis inhibitors that can be used in the methods and compositions of the invention include but are not limited to: Angiostatin (plasminogen fragment); antiangiogenic antithrombin III; Angiozyme; ABT-627; Bay 12-9566; Benefin; Bevacizumab; BMS-275291; cartilage-derived inhibitor (CDI); CAI; CD59 complement fragment; CEP-7055; Col 3; Combretastatin A-4; Endostatin (collagen XVIII fragment); Fibronectin fragment; Gro-beta; Halofuginone; Heparinases; Heparin hexasaccharide fragment; HMV833; Human chorionic gonadotropin (hCG); IM-862; Interferon alpha/beta/gamma; Interferon inducible protein (IP-10); Interleukin-12; Kringle 5 (plasminogen fragment); Marimastat; Metalloproteinase inhibitors (TIMPs); 2-Methoxyestradiol; MMI 270 (CGS 27023A); MoAb IMC-1C11; Neovastat; NM-3; Panzem; PI-88; Placental ribonuclease inhibitor; Plasminogen activator inhibitor; Platelet factor-4 (PF4); Prinomastat; Prolactin 16kD fragment; Proliferin-related protein (PRP); PTK 787/ZK 222594; Retinoids; Solimastat; Squalamine; SS 3304; SU 5416; SU6668; SU11248; Tetrahydrocortisol-S; tetrathiomolybdate; thalidomide; Thrombospondin-1 (TSP-1); TNP-470; Transforming growth factor-beta (TGF-b); Vasculostatin; Vasostatin (calreticulin fragment); ZD6126; ZD 6474; famesyl transferase inhibitors (FTI); and bisphosphonates.

Other anti-cancer agents that can be used in accordance with the methods of invention, include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukins (including recombinant interleukin 12, or rIL12, interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterium cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

Other examples of antineoplastic agents that may be administered in accordance with the methods of the invention include therapeutic antibodies including but not limited to ZENAPAX® (daclizumab) (Roche Pharmaceuticals, Switzerland) which is an immunosuppressive, humanized anti-CD25 monoclonal antibody for the prevention of acute renal allograft rejection; PANOREXTM™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (InClone System); VITAXIN™ which is a humanized anti-αVβ3 integrin antibody (Applied Molecular Evolution/MedImmune); Smart M195 which is a humanized anti-CD33 IgG antibody (Protein Design Lab/Kanebo); LYMPHOCIDE™ which is a humanized anti-CD22 IgG antibody (Immunomedics); ICM3 is a humanized anti-ICAM3 antibody (ICOS Pharm); IDEC-114 is a primatied anti-CD80 antibody (IDEC Pharm/Mitsubishi); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgGl antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech).

Particularly preferred biologically active agents that may be used in the instant invention are therapeutic antibodies. The invention encompasses monoclonal antibodies, multispecific antibodies, human antibodies, murine antibodies, humanized antibodies, synthetic antibodies, chimeric antibodies, polyclonal antibodies, camelized antibodies, single-chain Fvs (scFv), single chain antibodies, Fab fragments, F(ab′) fragments, disulfide-linked bispecific Fvs (sdFv), intrabodies, and anti-idiotypic (anti-Id) antibodies (including, e.g., anti-Id and anti-anti-Id antibodies to antibodies of the invention), and epitope-binding fragments of any of therapeutic antibodies disclosed herein and known in the art. Therapeutic antibodies encopassed by the invention include but are not limited to HERCEPTIN® (Trastuzumab) (Genentech, CA) which is a humanized anti-HER2 monoclonal antibody for the treatment of patients with metastatic breast cancer; REOPRO® (abciximab) (Centocor) which is an anti-glycoprotein IIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgG 1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primatied anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech). One skilled in the art will appreciate that the antibodies disclosed herein can be used prophylactically or preventatively to prevent or delay the onset or progression of a disease state, for example, cancer, tumor growth, metastasis of cancer, or infectious disease.

In some embodiments, the invention encompasses antibodies specific for a respiratory tract pathogen, for example, Parainfluenza, influenza A, influenza B, chlamydia or adenovirus. In preferred embodiments, the invention encompasses fully murine RSV specific antibodies (RSV48), humanized palivizumab or chimeric derivatives thereof.

Other examples of antibodies that can be used in accordance with the instant invention are listed in Table 1 below.

TABLE 1
Monoclonal antibodies for Cancer Therapy that
can be used in accordance with the invention.

| Company | Product | Disease | Target |
|---|---|---|---|
| Abgenix | ABX-EGF | Cancer | EGF receptor |
| AltaRex | OvaRex | ovarian cancer | tumor antigen CA125 |
| | BravaRex | Metastatic | tumor antigen MUC1 |
| | | cancers | |
| Antisoma | Theragyn | ovarian cancer | PEM antigen |
| | (pemtumomabytrrium- | | |
| | 90) | | |
| | Therex | breast cancer | PEM antigen |
| Boehringer | Blvatuzumab | head & neck | CD44 |
| Ingelheim | | cancer | |
| Centocor/J&J | Panorex | Colorectal | 17-1A |
| | | cancer | |
| | ReoPro | PTCA | gp IIIb/IIIa |
| | ReoPro | Acute MI | gp IIIb/IIIa |
| | ReoPro | Ischemic stroke | gp IIIb/IIIa |
| Corixa | Bexocar | NHL | CD20 |
| CRC | MAb, idiotypic 105AD7 | colorectal cancer | gp72 |
| Technology | | vaccine | |
| Crucell | Anti-EpCAM | Cancer | Ep-CAM |
| Cytoclonal | MAb, lung cancer | non-small cell | NA |
| | | lung cancer | |
| Genentech | Herceptin | metastatic breast | HER-2 |
| | | cancer | |
| | Herceptin | early stage | HER-2 |
| | | breast cancer | |
| | Rituxan | Relapsed/refractory | CD20 |
| | | low-grade or | |
| | | follicular NHL | |
| | Rituxan | intermediate & | CD20 |
| | | high-grade NHL | |
| | MAb-VEGF | NSCLC, | VEGF |
| | | metastatic | |
| | MAb-VEGF | Colorectal | VEGF |
| | | cancer, | |
| | | metastatic | |
| | AMD Fab | age-related | CD18 |
| | | macular | |
| | | degeneration | |
| | E-26 (2^{nd} gen. IgE) | allergic asthma | IgE |
| | | & rhinitis | |
| IDEC | Zevalin (Rituxan + | low grade of | CD20 |
| | yttrium-90) | follicular, | |
| | | relapsed or | |
| | | refractory, | |
| | | CD20-positive, | |
| | | B-cell NHL and | |
| | | Rituximab- | |
| | | refractory NHL | |
| ImClone | Cetuximab + innotecan | Refractory | EGF receptor |
| | | colorectal | |
| | | carcinoma | |
| | Cetuximab + cisplatin & | newly diagnosed | EGF receptor |
| | radiation | or recurrent head | |
| | | & neck cancer | |
| | Cetuximab + gemcitabine | newly diagnosed | EGF receptor |
| | | metastatic | |
| | | pancreatic | |
| | | carcinoma | |
| | Cetuximab + | recurrent or | EGF receptor |
| | cisplatin + | metastatic head | |
| | 5FU or Taxol | & neck cancer | |
| | Cetuximab + | newly diagnosed | EGF receptor |
| | carboplatin + | non-small cell | |
| | paclitaxel | lung carcinoma | |
| | Cetuximab + cisplatin | head & neck | EGF receptor |
| | | cancer | |
| | | (extensive | |
| | | incurable local- | |
| | | regional disease | |
| | | & distant | |
| | | metasteses) | |
| | Cetuximab + radiation | locally advanced | EGF receptor |
| | | head & neck | |
| | | carcinoma | |
| | BEC2 + Bacillus | small cell lung | mimics ganglioside |
| | Calmette Guerin | carcinoma | GD3 |
| | BEC2 + Bacillus | Melanoma | mimics ganglioside |
| | Calmette Guerin | | GD3 |
| | IMC-1C11 | colorectal cancer | VEGF-receptor |
| | | with liver | |
| | | metasteses | |
| ImmonoGen | nuC242-DM1 | Colorectal, | nuC242 |
| | | gastric, and | |
| | | pancreatic | |
| | | cancer | |
| ImmunoMedics | LymphoCide | Non-Hodgkins | CD22 |
| | | lymphoma | |
| | LymphoCide Y-90 | Non-Hodgkins | CD22 |
| | | lymphoma | |
| | CEA-Cide | metastatic solid | CEA |
| | | tumors | |
| | CEA-Cide Y-90 | metastatic solid | CEA |
| | | tumors | |
| | CEA-Scan (Tc-99m- | colorectal cancer | CEA |
| | labeled arcitumomab) | (radioimaging) | |
| | CEA-Scan (Tc-99m- | Breast cancer | CEA |
| | labeled arcitumomab) | (radioimaging) | |
| | CEA-Scan (Tc-99m- | lung cancer | CEA |
| | labeled arcitumomab) | (radioimaging) | |
| | CEA-Scan (Tc-99m- | Intraoperative | CEA |
| | labeled arcitumomab) | tumors (radio | |
| | | imaging) | |
| | LeukoScan (Tc-99m- | soft tissue | CEA |
| | labeled sulesomab) | infection | |
| | | (radioimaging) | |
| | LymphoScan (Tc-99m- | Lymphomas | CD22 |
| | labeled) | (radioimaging) | |
| | AFP-Scan (Tc-99m- | liver 7 gem-cell | AFP |
| | labeled) | cancers | |
| | | (radioimaging) | |
| Intracel | HumaRAD-HN (+ | head & neck | NA |
| | yttrium-90) | cancer | |
| | HumaSPECT | Colorectal | NA |
| | | imaging | |
| Medarex | MDX-101 (CTLA-4) | Prostate and | CTLA-4 |
| | | other cancers | |
| | MDX-210 (her-2 | Prostate cancer | HER-2 |
| | overexpression) | | |
| | MDX-210/MAK | Cancer | HER-2 |
| MedImmune | Vitaxin | Cancer | αvβ₃ |
| Merck KGaA | MAb 425 | Various cancers | EGF receptor |
| | IS-IL-2 | Various cancers | Ep-CAM |
| Millennium | Campath (alemtuzumab) | Chronic | CD52 |
| | | lymphocytic | |
| | | leukemia | |
| NeoRx | CD20-streptavidin (+ | Non-Hodgkins | CD20 |
| | biotin-yttrium 90) | lymphoma | |
| | Avidicin (albumin + | Metastatic | NA |
| | NRLU13) | cancer | |
| Peregrine | Oncolym (+ iodine-131) | Non-Hodgkins | HLA-DR 10 beta |
| | | lymphoma | |
| | Cotara (+ iodine-131) | Unresectable | DNA-associated |
| | | malignant | proteins |
| | | glioma | |
| Pharmacia | C215 (+ staphylococcal | Pancreatic | NA |
| Corporation | enterotoxin) | cancer | |
| | MAb, lung/kidney cancer | lung & kidney | NA |
| | | cancer | |
| | nacolomab tafenatox | colon & | NA |
| | (C242 + staphylococcal | pancreatic | |
| | enterotoxin) | cancer | |
| Protein Design | Nuvion | T cell | CD3 |
| Labs | | malignancies | |
| | SMART M195 | AML | CD33 |
| | SMART 1D10 | NHL | HLA-DR antigen |
| Titan | CEAVac | Colorectal | CEA |
| | | cancer, | |
| | | advanced | |
| | TriGem | Metastatic | GD2-ganglioside |
| | | melanoma & | |
| | | small cell lung | |
| | | cancer | |
| | TriAb | metastatic breast | MUC-1 |
| | | cancer | |
| Trilex | CEAVac | Colorectal | CEA |
| | | cancer, | |
| | | advanced | |
| | TriGem | Metastatic | GD2-ganglioside |
| | | melanoma & | |
| | | small cell lung | |
| | | cancer | |
| | TriAb | metastatic breast | MUC-1 |
| | | cancer | |
| Viventia | NovoMAb-G2 | Non-Hodgkins | NA |
| Biotech | radiolabeled | lymphoma | SK-1 antigen |
| | Monopharm C | colorectal & | |
| | | pancreatic | |
| | | carcinoma | |
| | GlioMAb-H (+ | gliorna, | NA |
| | gelonin toxin) | melanoma & | |
| | | neuroblastoma | |
| Xoma | Rituxan | Relapsed/refractory | CD20 |
| | | low-grade or | |
| | | follicular NHL | |
| | Rituxan | intermediate & | CD20 |
| | | high-grade NHL | |
| | ING-1 | Adenomcarcinoma | Ep-CAM |

Therapeutic agents that may be used in the compositions of the invention include but are not limited to chemotherapeutic agents, radiation therapeutic agents, hormonal therapeutic agents, immunotherapeutic agents, immunomodulatory agents, anti-inflammatory agents, antibiotics, anti-viral agents, and cytotoxic agents.

Non-limiting examples of anti-inflammatory agents include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

Examples of immunomodulatory agents include, but are not limited to, methothrexate, ENBREL, REMICADE™, leflunomide, cyclophosphamide, cyclosporine A, and macrolide antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunamide), T cell receptor modulators, and cytokine receptor modulators, corticosteroids, cytokine agonists, cytokine antagonists, and cytokine inhibitors.

Examples of antibiotics include, but are not limited to, macrolide (e.g., tobramycin (Tobi®)), a cephalosporin (e.g., cephalexin (Keflex®), cephradine (Velosef®), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)),aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin, chloramphenicols, erythromycin, penicillin, streptomycin, vancomycin, trimethoprimsulfamethoxazols, and tuberin.

Examples of anti-viral agents include, but are not limited to, protease inhibitors, nucleoside reverse transcriptase inhibitors, non-nucleoside reverse transcriptase inhibitors and nucleoside analogs, zidovudine, acyclovir, gangcyclovir, vidarabine, idoxuridine, trifluridine, and ribavirin, as well as foscarnet, amantadine, rimantadine, saquinavir, indinavir, amprenavir, lopinavir, ritonavir, the alpha-interferons; adefovir, clevadine, entecavir, and pleconaril, Ribavirin, rimantadine, Amantadine, neuraminidase inhibitors and numerous lipid derivitized drugs, natural fatty acids, phospholipids or docosahexaenoic acid (DHA).

Other therapeutic agents which can be used with the present invention include but are not limited to Alpha-1 anti-trypsin, Anti-Angiogenesis agents, Antisense, butorphanol, Calcitonin and analogs, Ceredase, COX-II inhibitors, dermatological agents, dihydroergotamine, Dopamine agonists and antagonists, Enkephalins and other opioid peptides, Epidermal growth factors, Erythropoietin and analogs, Follicle stimulating hormone, G-CSF, Glucagon, GM-CSF, granisetron, Growth hormone and analogs (including growth hormone releasing hormone), Growth hormone antagonists, Hirudin and Hirudin analogs such as Hirulog, IgE suppressors, Insulin, insulinotropin and analogs, Insulin-like growth factors, Interferons, Interleukins, Luteinizing hormone, Luteinizing hormone releasing hormone and analogs, Heparins, Low molecular weight heparins and other natural, modified, or synthetic glycoaminoglycans, M-CSF, metoclopramide, Midazolam, Monoclonal antibodies, Pegylated antibodies, Pegylated proteins or any proteins modified with hydrophilic or hydrophobic polymers or additional functional groups, Fusion proteins, Single chain antibody fragments or the same with any combination of attached proteins, macromolecules, or additional functional groups thereof, Narcotic analgesics, nicotine, Non-steroid anti-inflammatory agents, Oligosaccharides, ondansetron, Parathyroid hormone and analogs, Parathyroid hormone antagonists, Prostaglandin antagonists, Prostaglandins, Recombinant soluble receptors, scopolamine, Serotonin agonists and antagonists, Sildenafil, Terbutaline, Thrombolytics, Tissue plasminogen activators, TNF, and TNF antagonist, the vaccines, with or without carriers/adjuvants, including prophylactics and therapeutic antigens (including but not limited to subunit protein, peptide and polysaccharide, polysaccharide conjugates, toxoids, genetic based vaccines, live attenuated, reassortant, inactivated, whole cells, viral and bacterial vectors) in connection with, addiction, arthritis, cholera, cocaine addiction, diphtheria, tetanus, HIB, Lyme disease, meningococcus, measles, mumps, rubella, varicella, yellow fever, Respiratory syncytial virus, tick borne japanese encephalitis, pneumococcus, streptococcus, typhoid, influenza, hepatitis, including hepatitis A, B, C and E, otitis media, rabies, polio, HIV, parainfluenza, rotavirus, Epstein Barr Virus, CMV, chlamydia, non-typeable haemophilus, moraxella catarrhalis, human papilloma virus, tuberculosis including BCG, gonorrhoea, asthma, atheroschlerosis malaria, E-coli, Alzheimer's Disease, H. Pylori, salmonella, diabetes, cancer, herpes simplex, human papilloma and the like other substances including all of the major. therapeutics such as agents for the common cold, Anti-addiction, anti-allergy, anti-emetics, anti-obesity, antiosteoporeteic, anti-infectives, analgesics, anesthetics, anorexics, antiarthritics, antiasthmatic agents, anticonvulsants, antidepressants, antidiabetic agents, antihistamines, anti-inflammatory agents, antimigraine preparations, antimotion sickness preparations, antinauseants, antineoplastics, antiparkinsonism drugs, antipruritics, antipsychotics, antipyretics, anticholinergics, benzodiazepine antagonists, vasodilators, including general, coronary, peripheral and cerebral, bone stimulating agents, central nervous system stimulants, hormones, hypnotics, immunosuppressives, muscle relaxants, parasympatholytics, parasympathomimetrics, prostaglandins, proteins, peptides, polypeptides and other macromolecules, psychostimulants, sedatives, and sexual hypofunction and tranquilizers.

5.3.1 Compositions

The invention encompasses compositions (or formulations) comprising one or more biologically active agents, particularly therapeutic agents, in solution forms, particulate forms thereof and mixtures thereof. Compositions for use in the methods of the invention may be obtained from any species or generated by any recombinant DNA technology known to one skilled in the art. Compositions comprising one or more biologically active agents may be from different animal species including, limited but not to, swine, bovine, ovine, equine, etc. The chemical state of such agents may be modified by standard recombinant DNA technology to produce agents of different chemical formulas in different association states.

The form of the biologically active agent to be delivered or administered include solutions thereof in pharmaceutically acceptable diluents or solvents, emulsions, suspensions, gels, particulates such as micro- and nanoparticles either suspended or dispersed, as well as in-situ forming vehicles of the same. The compositions of the invention may be in any form suitable for intradermal delivery. In one embodiment, the intradermal composition of the invention is in the form of a flowable, injectible medium, i.e., a low viscosity composition that may be injected in a syringe or pen. The flowable injectible medium may be a liquid. Alternatively the flowable injectible medium is a liquid in which particulate material is suspended, such that the medium retains its fluidity to be injectible and syringable, e.g., can be administered in a syringe.

In some embodiments, the formulations of the invention comprise a therapeutically effective amount of an agent and one or more other additives. Additives that may be used in the formulations of the invention include for example, wetting agents, emulsifying agents, agents that change the quaternary structure of insulin or pH buffering agents. The formulations of the invention may contain one or more other excipients such as saccharides and polyols. Additional examples of pharmaceutically acceptable carriers, diluents, and other excipients are provided in Remington's Pharmaceutical Sciences (Mack Pub. Co. N.J. current edition, all of which is incorporated herein by reference in its entirety.

The form of the therapeutic agent to be delivered or administered include solutions thereof in pharmaceutically acceptable diluents or solvents, emulsions, suspensions, gels, particulates such as micro- and nanoparticles either suspended or dispersed, as well as in-situ forming vehicles of the same. The formulations of the invention may be in any form suitable for intradermal delivery. In one embodiment, the intradermal formulation of the invention is in the form of a flowable, injectible medium, i.e., a low viscosity formulation that may be injected in a syringe or insulin pen. The flowable injectible medium may be a liquid. Alternatively the flowable injectible medium is a liquid in which particulate material is suspended, such that the medium retains its fluidity to be injectible and syringable, e.g., can be administered in a syringe.

The intradermal formulations of the present invention can be prepared as unit dosage forms. A unit dosage per vial may contain 0.1 to 0.5 mL of the formulation. In some embodiments, a unit dosage form of the intradermal formulations of the invention may contain 50 μL to 100 μL, 50 μL to 200 μL, or 50 μL to 500 μL of the formulation. If necessary, these preparations can be adjusted to a desired concentration by adding a sterile diluent to each vial. Formulations administered in accordance with the methods of the invention are not administered in volumes whereby the intradermal space might become overloaded leading to partitioning to one or more other compartments, such as the SC compartment.

The therapeutic agents used in the methods of the invention may be in liquid or powder form. In specific embodiments, where a formulation is administered intranasallty, the liquid form would include the therapeutic agent, e.g., antibody, given as drops or aerosolized.

The powder form of the therapeutic agent would include powders prepared by any of a variety of methods known in the art, for example lyophilization, spray drying, or spray-freeze drying (SFD) methods, as described for example in U.S. application Ser. No. 10/299,012, which is incorporated herein by reference in its entirety.

Powder forms of the agents may comprise purely of the therapeutic agent or alternatively may contain one or more additional components. In general, a therapeutic agent of interest can be initially formulated as a liquid formulation, using any of a variety of conventional liquids. Preferably, the liquid is an aqueous one, such as, e.g., water (e.g., injectable grade water) or any of a variety of conventional buffers, which may or may not contain salts. The pH of the buffer will generally be chosen to stabilize the protein or other type of therapeutic agent of choice, and will be ascertainable by those in the art. Generally, this will be in the range of physiological pH, although some proteins can be stable at a wider range of pHs, for example acidic pH. Thus, preferred pH ranges of the initial liquid formulation are from about 1 to about 10, with from about 3 to about 8 being particularly preferred, and from about 5 to about 7 being especially preferred. As will be appreciated by those in the art, there are a large number of suitable buffers that may be used. Suitable buffers include, but are not limited to, sodium acetate, sodium citrate, sodium succinate, ammonium bicarbonate and carbonate. Generally, buffers are used at molarities from about 1 mM to about 2 M, with from about 2 mM to about 1 M being preferred, and from about 10 mM to about 0.5 M being especially preferred, and 50 to 200 mM being particularly preferred. Generally, salts, if present in the liquid solution, are used at molarities from about 1 mM to about 2 M, with from about 2 mM to about 1 M being preferred, and from about 10 mM to about 0.5 M being especially preferred, and 50 to 200 mM being particularly preferred. Suitable salts include, but are not limited to, NaCl.

The liquid formulation can be in any of a variety of forms, e.g., a solution, a suspension, an emulsion, such as a oil/water or water/oil/water emulsion, a slurry or a colloid.

Optionally, the liquid formulation can comprise one or more conventional pharmaceutically acceptable excipients. “Excipients” generally refer to compounds or materials that are added to enhance the efficacy of a formulation of an active pharmaceutical ingredient (API). Examples include, e.g., cryoprotectants and lyoprotectants, which are added to ensure or increase the stability of the protein during the spray-freeze dry process or spray-freeze atmosphere dry process, and afterwards, for long term stability and flowability of the powder product. Suitable protectants are generally relatively free flowing particulate solids, do not thicken or polymerize upon contact with water, are essentially innocuous when inhaled by a patient or otherwise introduced into a patient, and do not significantly interact with the therapeutic agent in a manner that alters its biological activity. Suitable excipients include, but are not limited to, proteins such as human and bovine serum albumin, gelatin, immunoglobulins, carbohydrates including monosaccharides (e.g.,galactose, D-mannose, sorbose, etc.), disaccharides (e.g.,lactose, trehalose, sucrose, etc.), cyclodextrins, and polysaccharides (e.g.,raffinose, maltodextrins, dextrans, etc.); an amino acid such as monosodium glutamate, glycine, alanine, arginine or histidine, as well as hydrophobic amino acids (e.g.,tryptophan, tyrosine, leucine, phenylalanine, etc.); a methylamine such as betaine; an excipient salt such as magnesium sulfate; a polyol such as trihydric or higher sugar alcohols, e.g., glycerin, erythritol, glycerol, arabitol, xylitol, sorbitol, and mannitol; propylene glycol; polyethylene glycol; Pluronics; surfactants; and combinations thereof. Preferred excipients include e.g., trehalose, sucrose and mannitol. Another class of excipient, mucoadhesives, are often used to increase contact of an API with mucosal surfaces. Examples of mucoadhesives include, e.g., chitosan, dermatan sulfate, chondroitin, and pectin. Additionally, conventional cosolvents, which improve the solubility of APIs, can be added to liquid formulations suitable for the SFD processes disclosed herein.

Generally, when mucoadhesives are used, they are used in amounts ranging from about 1 to 95 wt %, with from about 1 to 50 wt % preferred, from about 5 to 50 wt % being especially preferred, and from about 5 to 20% being particularly preferred. In general, cryoprotectants are used at a concentration of between about 5 wt % and about 95 wt %.

The dried powders of the invention can be combined with bulking agents or carriers, which are used to reduce the concentration of the therapeutic agent in the powder being delivered to a patient; that is, it may be desirable to have larger volumes of material per unit dose. Bulking agents may also be used to improve the handling characteristics of the powder. Suitable bulking agents are generally crystalline (to avoid water absorption) and include, but are not limited to, lactose and mannitol. Accordingly, bulking agents such as lactose, if added, may be added in varying ratios, with from about 99:1 of a therapeutic agent of interest to bulking agent to about 1:99 being preferred, and from about 1:5 to about 5:1 being more preferred, and from about 1:10 to about 1:20 being especially preferred.

The invention encompasses administering the compositions of the invention intradermally as disclosed herein in combination with other routes of delivery including for example, subcutaneous-intradermal interface, intransal (IN), parenteral administration (e.g., intramuscular, intraperitoneal, intravenous and subcutaneous), epidural, and mucosal (e.g., intranasal and oral routes), intratumoral, peritumoral, topical, and epidermal. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

5.3.2 Characterization of Therapeutic Utility

Several aspects of the compositions, prophylactic, or therapeutic agents of the invention are preferably tested in vitro, in a cell culture system, and in an animal model organism, such as a rodent animal model system, for the desired therapeutic activity prior to use in humans. For example, assays which can be used to determine whether administration of a specific composition is desired, include cell culture assays in which a patient tissue sample is grown in culture, and exposed to or otherwise contacted with a pharmaceutical composition of the invention, and the effect of such composition upon the tissue sample is observed. The tissue sample can be obtained by biopsy from the patient. This test allows the identification of the therapeutically most effective prophylactic or therapeutic molecule(s) for each individual patient. In various specific embodiments, in vitro assays can be carried out with representative cells of cell types involved in an autoimmune or inflammatory disorder (e.g., T cells), to determine if a pharmaceutical composition of the invention has a desired effect upon such cell types.

Combinations of prophylactic and/or therapeutic agents can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, combinations of prophylactic and/or therapeutic agents are tested in a mouse model system. Such model systems are widely used and well-known to the skilled artisan. Prophylactic and/or therapeutic agents can be administered repeatedly. Several aspects of the procedure may vary. Said aspects include the temporal regime of administering the prophylactic and/or therapeutic agents, and whether such agents are administered separately or as an admixture.

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

the data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

The anti-cancer activity of the therapies used in accordance with the present invention also can be determined by using various experimental animal models for the study of cancer such as the SCID mouse model or transgenic mice or nude mice with human xenografts, animal models, such as hamsters, rabbits, etc. known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher), herein incorporated by reference in their entireties.

Therapeutic agents and methods may be screened using cells of a tumor or malignant cell line. Many assays standard in the art can be used to assess such survival and/or growth; for example, cell proliferation can be assayed by measuring ³H-thymidine incorporation, by direct cell count, by detecting changes in transcriptional activity of known genes such as proto-oncogenes (e.g., fos, myc) or cell cycle markers; cell viability can be assessed by trypan blue staining, differentiation can be assessed visually based on changes in morphology, decreased growth and/or colony formation in soft agar or tubular network formation in three-dimensional basement membrane or extracellular matrix preparation, etc.

Further, any assays known to those skilled in the art can be used to evaluate the prophylactic and/or therapeutic utility of the combinatorial therapies disclosed herein for treatment or prevention of cancer, inflammatory disorder, or autoimmune disease.

### 6. EXAMPLES

6.1 Effects of Routes of Delivery on Antitumor Activity of IL-12 Using 100 ng Dose

6.1.1 Procedures

C57BL/6J mice (Charles Rivers Labs, Inc) were inoculated SC with 1×10⁶ B 16F10 melanoma cells (ATCC) on the upper right flank. One week following tumor inoculation (Day 7), the mice were randomized and treated with 100 ng rmIL-12 (R&D Systems) in 50 μL PBS solution through either ID or SC injections near tumor inoculation site, or an IP injection (n=25/condition). An additional condition of ID controls was run using 50 μL PBS solution alone. ID treatment was administered with a 34G, 1 mm needle using a modified mantoux method. SC and IP doses were given with standard 25G×¾″ needle. Treatment was continued every other day through Day 13 for a total of four doses. Tumor measurements in mm² (length×width) were collected on Days 7, 11, 14, 18, 21, 25 and 28. Blood samples were collected on Days 0, 14, 21 and 28 for IL-12 and IFN-gamma analysis. Draining lymph node and spleen tissue samples were collected from five animals in each condition on Days 14 and 24 for FACS analysis of CD49b (NK) cells. Mortality data were collected on 10 pre-selected animals that were not used for blood sampling or FACS analysis from each condition. Mortality was determined through either natural death or euthanasia due to tumor size of greater than 400 mm². Statistical data were calculated using a t-Test: Two-Sample Assuming Unequal Variances. See, e.g., Brunda, M., J. Exp. Med., 178: 1223-1230 (1993) and Leonard et al., Blood, 90: 2541-2548 (1997), both of which are incorporated herein by reference.

6.1.2 FACS Analysis

The right side superficial inguinal draining lymph node (DLN) and the spleen were excised individually from 5 mice per each treatment group into Petri dishes containing cold HBSS (Invitrogen Life Technologies, Carlsbad, Calif.) for the DLN. Spleens were placed in 10 ml cold red blood cell lysis buffer (0.16M NH₄Cl and 10 mM KHCO₃, Sigma, St. Louis, Mo.). Each DLN and spleen was processed into single cell suspensions by mechanical disruption. Cell counts were taken using a 1:20 dilution from the resulting cell solution. Cells were centrifuged at 1500 rpm for 15 minutes at 4° C. The supernatant was aspirated and the cells were washed once with 5 ml HBSS buffer and centrifuged once again using the same condition. The supernatant was aspirated, and the cells were resuspended in Pharmingen stain buffer (Pharmingen, BD Biosciences, San Jose, Calif.) at 2-4 ×⁸ cells/ml for flow staining. Approximately 1×10⁷ cells (25 μl) of resuspended cells were added to a well of a 96 well plate. Staining cocktail (25μl) was added to the cells in the well and mixed by pipetting. The cocktail consisted of a combination of the following labeled antibodies, as appropriate, each at 0.01 mg/ml in Pharmingen stain buffer: FITC-CD49b (Pan-NK cell, clone DX5, Pharmingen, BD Biosciences, San Jose, Calif.); PE-CD19 (Pan B cell, clone 1D3, Pharmingen, BD Biosciences, San Jose, Calif.); CY5PE-B220 (granulocytes and monocytes, clone RA3-6B2, Pharmingen, BD Biosciences, San Jose, Calif.); APC-CD4 (T helper cell, clone RM4-5, Pharmingen, BD Biosciences, San Jose, Calif.); and APC-CY7-CD8 (T cytotoxic cell, clone BC-CD8a, Biocarta, San Diego, Calif.). The cell/stain mix was incubated for 1 hour at 4° C. in the dark. The wells were washed with 150 μl FacsFlow buffer (Pharmingen, BD Biosciences, San Jose, Calif.), and centrifuged at 1500 rpm for 5 minutes at 4° C. The supernatant was aspirated and the wash was repeated. The washed cells were resuspended in 1 ml of cold FacsFlow buffer and kept on ice in the dark until analyzed by flow cytometry using a FACS Vantage SE. Cell analysis was designed to exclude B cells and quantify CD4+ cells, CD8+ cells, and CD49b+ (NK cells). See, e.g., Cordaro, T., J. Immunology, 168: 651-660 (2002); Fogler et al., J. Immonology, 161: 6014-6021 (1998); Gao et al., J. Exp. Med., 198(3): 4330442 (2003); Harada et al., Int. J. Cancer, 75: 400-405 (1998); Jenne et al., Cancer Research, 60: 4446-4452 (2000); and Park et al., J. Immunology, 170: 1197-1201 (2003), all of which are incorporated herein by reference.

6.1.3 Tumor Growth

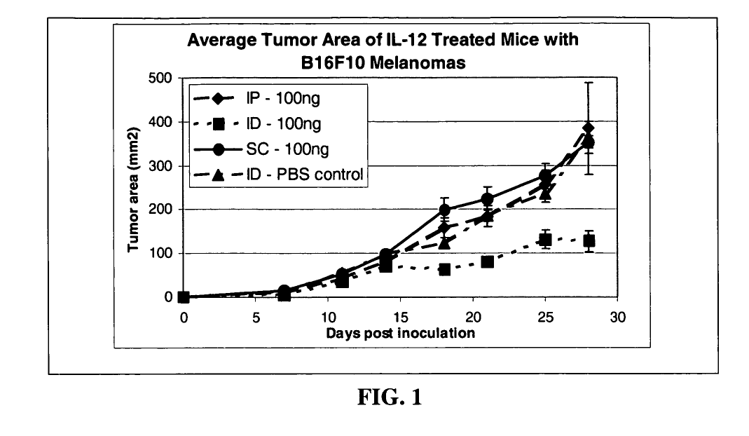

As shown in FIG. 1, only IL-12, delivered ID, showed significant effects on inhibiting the tumor growth. A significant (p<0.0002) decrease in tumor size was seen in the ID dosed condition over IP delivery at Day 18. A similar effect was seen over the ID-delivered PBS (p<0.0000005) and the SC (p<0.00003) conditions. The significant trend followed through the end of the study, i.e., Day 28.

The dose of IL-12 in this study was 2-10 times lower than effective IP doses disclosed in literature. See, e.g., Tannenbaum et al., J. Immunology, 156: 693-699 (1996) and Tsung et al., J. Immunology, 158: 3359-3365 (1997). This may account for the lack of response seen in this study to IP dosing with IL-12. This shows a dose sparing effect over other published treatments. Lower doses by directly targeting the lymphatics would mean reduced cost, and suggest a probable decrease in side effects.

6.1.4 Mortality

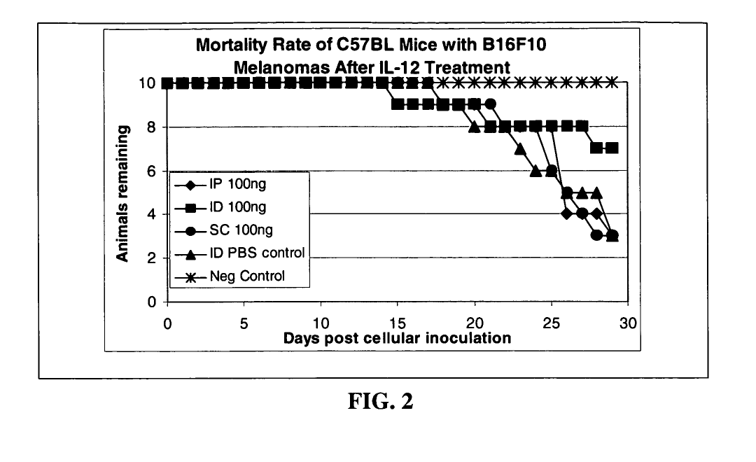

As shown in FIG. 2, at Day 28, mortality was lower for the group that received IL-12 through ID delivery (7 out of 10 surviving), than those of other groups, which received IL-12 through IP (3 out of 10 surviving) or SC (3 out of 10 surviving) delivery. The mortality shown for the group that received IL-12 through ID delivery was also lower than that of the group that received PBS only through ID delivery (3 out of 10 surviving). Thus, ID delivery of IL-12 shows a greater than 200% survivability at Day 28 compared to delivery through other routes or control.

6.1.5 NK Cells Count

IFN-γ is thought to be the curative action of IL-12 delivery. IL-12 has been shown to increase IFN-γ production from NK and T-cells, and to promote expansion and differentiation of NK cells. Thus the increase in NK cells is indicative of an enhanced efficacy of IL-12 therapy. Without being limited by a particular theory, the increase in NK cells could be caused by: enhanced NK cell proliferation or differentiation; enhanced IFN-gamma production and release; greater targeting to the site of tumor propagation; better efficacy through effective targeting of immunomodulatory processes via a lymphatic uptake pathway; and/or combinations of the above.

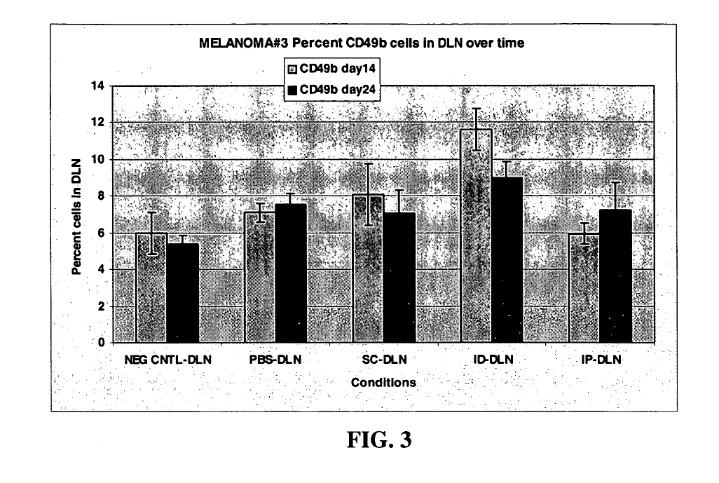

As shown in FIG. 3, the increase in NK cells is apparent for the ID delivery group over the other conditions one day post IL-12 therapy regimen (Day 14) with a relative decrease at day 24 within the ID group, but still showing a relative increase above the other conditions at Day 24.

6.1.6 Statistical Data

The following statistical data obtained from t-tests on Days 18 and 28 show the significance of ID delivery of IL-12 over all other tested deliveries.

TABLE 1
T-Test Data on Day 18
IP v. ID

| | IP 100 ng | ID 100 ng |
|---|---|---|
| Mean | 158.2458 | 62.28816 |
| Variance | 8780.02 | 2255.463 |
| Observations | 19 | 19 |
| Hypothesized Mean Difference | 0 | |
| Df | 27 | |
| t Stat | 3.981629 | |
| P (T <= t) one-tail | 0.000232 | |
| t Critical one-tail | 1.703288 | |
| P (T <= t) two-tail | 0.000465 | |
| t Critical two-tail | 2.051829 | |

ID v. SC

| | ID 100 ng | SC 100 ng |
|---|---|---|
| Mean | 62.288163 | 199.7379 |
| Variance | 2255.4628 | 12576.68 |
| Observations | 19 | 18 |
| Hypothesized Mean Difference | 0 | |
| Df | 23 | |
| t Stat | −4.807544 | |
| P (T <= t) one-tail | 3.761E−05 | |
| t Critical one-tail | 1.71387 | |
| P (T <= t) two-tail | 7.523E−05 | |
| t Critical two-tail | 2.0686548 | |

ID v. Control

| | ID 100 ng | ID-PBS |
|---|---|---|
| Mean | 62.288163 | 122.8243 |
| Variance | 2255.4628 | 3168.4 |
| Observations | 19 | 19 |
| Hypothesized Mean Difference | 0 | |
| Df | 35 | |
| t Stat | −3.582918 | |
| P (T <= t) one-tail | 0.0005116 | |
| t Critical one-tail | 1.6895729 | |
| P (T <= t) two-tail | 0.0010233 | |
| t Critical two-tail | 2.0301104 | |

TABLE 2
T-Test Data on Day 28
IP v. ID

| | IP 100 ng | ID 100 ng |
|---|---|---|
| Mean | 383.69215 | 127.1987286 |
| Variance | 43458.96777 | 4093.474314 |
| Observations | 4 | 7 |
| Hypothesized Mean Difference | 0 | |
| Df | 3 | |
| t Stat | 2.397079927 | |
| P (T <= t) one-tail | 0.04806332 | |
| t Critical one-tail | 2.353363016 | |
| P (T <= t) two-tail | 0.09612664 | |
| t Critical two-tail | 3.182449291 | |

ID v. SC

| | ID 100 ng | SC 100 ng |
|---|---|---|
| Mean | 127.1987286 | 352.86435 |
| Variance | 4093.474314 | 762.4257392 |
| Observations | 7 | 4 |
| Hypothesized Mean Difference | 0 | |
| Df | 9 | |
| t Stat | −8.10411752 | |
| P (T <= t) one-tail | 9.97966E−06 | |
| t Critical one-tail | 1.833113856 | |
| P (T <= t) two-tail | 1.99593E−05 | |
| t Critical two-tail | 2.262158887 | |

ID v. Control

| | ID 100 ng | ID-PBS |
|---|---|---|
| Mean | 127.1987286 | 363.97564 |
| Variance | 4093.474314 | 6557.946662 |
| Observations | 7 | 5 |
| Hypothesized Mean Difference | 0 | |
| Df | 7 | |
| t Stat | −5.43722939 | |
| P (T <= t) one-tail | 0.000484554 | |
| t Critical one-tail | 1.894577508 | |
| P (T <= t) two-tail | 0.000969107 | |
| t Critical two-tail | 2.36462256 | |

6.2 Effects of ID and IP Delivery on Antitumor Activity of IL-12

C57BL Mice were inoculated subcutaneously (SC) with 1 million B16F10 melanoma cells (ATCC, Inc.). Treatment was initiated with IL-12 injections 7 days post-inoculation at two levels (10 and 100 ng) given as either an intraperitoneal (IP) or intradermal (ID) (tumor bearing side) dose. IL-12 doses were repeated every other day for a total of 4 treatments. As a control, the same volume of complete medium (PBS) was injected ID on the tumor bearing side. True negatives were also run in conjunction for serum and tissue samples (n=15/condition). Weights and tumor size were measured on days 7, 11, 14, 18 and 21. Blood samples were also collected at weekly intervals for IL-12 analysis. Two tumor diameters at right angles were measured with digital calipers, and the average tumor size was plotted with respect to days post inoculation with tumor cells.

6.2.1 Effects of ID and IP Delivery on Tumor Size at 10 ng IL-12 Dose

The growth rates were similar in the ID (77 mm²) dosed condition as compared to the IP (68 mm²) dosed condition at the first measurement post treatment, Day 11. At Day 14, the tumors of the ID treated mice increased 56% to 120 mm² while the tumors of the IP treated mice increased 63% from the previous measurement to 113 mm^{2.} Both treatment groups showed similar tumor sizes to PBS treatment which yielded an average tumor size of 74 mm² and 126 mm² (70%) for Days 11 and 14, respectively. However, at Day 18, the tumors of the ID treated mice decreased 6% (112 mm²), while those of the IP treated mice increased 82% (206 mm²). At Day 21, the ID condition increased 121% to 250 mm², whereas the IP condition increased 27% to 262 mm^{2.} The average tumor sizes for the ID and IP condition were similar to the PBS dosed animals at Day 21, 259 mm^{2.} The advantage of ID delivery over IP was only seen at Day 18 with the 10 ng dose of IL-12. The IL-12 injections ended on Day 13, the advantage may be more significant if treatment was given over broader or more frequent dosing schedule.

6.2.2 Effects of ID and IP Delivery on Tumor Size at 100 ng IL-12 Dose

An average reduced growth rate was seen in the ID (54 mm²) as compared to the IP (70 mm²) dosed conditions at the first measurement post treatment, Day 11. At Day 14, the tumors of the ID treated mice increased 39% to 75 nm² while the tumors of the IP treated mice increased 80% from the previous measurement to 126 mm². Only the ID treatment groups showed smaller tumor size and growth rate over PBS treatment which yielded an average tumor size of 74 mm² and 126 mm² (70%) for Days 11 and 14 respectively. By day 21, the ID condition increased 89% to 142 mm², where as the IP condition increased 139% to 302 mm^{2 .} For the 100 ng dose of IL-12, the average tumor growth rate was reduced with the ID condition over IP and the final average ID tumor size was 47% smaller than IP.

The difference in tumor size between the ID-treated group and the IP-treated group suggests that a lower dose can be administered ID than IP for an equal or better systemic response—a dose sparing and increased efficacy. A reduced injection amount should also decrease the amount and severity of side effects seen in higher doses. Lower tumor burden may also mean lower metastatic activity and increased survival rate.

6.3 Effects of Site of ID Delivery on Antitumor Activity of IL-12

C57BL Mice were inoculated subcutaneously (SC) with 1 million B16F10 melanoma cells (ATCC, Inc.). Treatment was initiated with IL-12 injections 7 days post-inoculation at 100 ng given as an ID dose on the tumor bearing or contralateral side dose. IL-12 doses were repeated every other day for a total of 4 treatments. As a control, the same volume of complete medium (PBS) was injected ID on the tumor bearing side. True negatives were also run in conjunction for serum and tissue samples (n=15/condition). Weights and tumor size were measured on days 7, 11, 14, 18 and 21. Blood samples were also collected at weekly intervals for IL-12 analysis. Draining lymph node and spleen samples were collected at 14 and 24 days for T cell, B cell and NK cell, and GP100 FACS analysis was performed. Two tumor diameters at right angles were measured with digital calipers, and the average tumor size was plotted with respect to days post inoculation with tumor cells.

6.3.1 Results

Significant differences in tumor size between the ID condition dosed next to the tumor and the ID dosed on the contralateral side first appeared at Day 18. From 75 mm² for each condition at Day 14, tumor for which the contralateral side injection (ID_{cs},) was performed increased 91% (to 43 mm²), while the tumor for which tumor side injection (IDₜₛ,) was performed condition increased only 68% to 126 mm². By Day 21, the ID_{cs}, increased another 29%, while the IDₜₛ, increased only 14% to 142 mm². Both ID conditions were significant improvements over PBS treated animals that had an average tumor size of 259 mm² at Day 21. The final tumor size for the IDₜₛ, condition was 23% smaller than the ID_{cs}, demonstrating the advantage of targeting the lymphatics directly associated with the tumor.

Injections of 100 ng IL-12 administered intradermally near the tumor injection site had the greatest reduction in overall tumor growth. Without being limited by a particular theory, this demonstrates the ability of ID injections to more efficiently target the tumor draining lymph nodes—the system involved in tumor trafficking, metastasis and immunologic response, than the other conditions. The ID injections of IL-12 given on the contralateral side were not as effective as those dosed near the tumor, but were better than the IP condition. Without being limited by a particular theory, this may be due to an immunological response still being achieved by directly targeting the lymphatics, but not accessing the direct lymphatics involved with the tumor.

6.4 Targeting Lung Tissue Using ID Delivery

Intramuscular (IM) and ID delivery routes were compared by measuring tissue and circulatory levels of RSV specific monoclonal antibody 48 (RSV MAB 48). RSV MAB 48 was generated by immunizing Balb/c mice with a purified RSV fusion protein. Primed B-cells were collected and fused with 653 myeloma line via PEG method. Other suitable monoclonal antibodies can be obtained by those of ordinary skill in the art using methods well-known in the art.

Lung tissue was collected and assayed for presence of antibody. Lung samples were collected at 3 hours, 24 hours, 7, 14, 21 and 28 days post-injection. To accomplish the desired sampling, the study was conducted in six stages. Two repetitions or two animals were designated for each tissue collection time and route so that two samples were provided for harvest at each time point.

6.4.1 Detailed Procedures

6.4.1.1 Preparation of Lung Tissue Lysates for ELISA

Lungs were harvested, rinsed in NaCl 0.9%, frozen at −20° C., and stored in a Vacutainer® (5 ml) at −20° C. until assayed. Before lysis, the tissue was thawed, and Hanks' Balanced Salt Solution (4 ml) and a transfer pipette were used to thoroughly rinse any remaining blood from the lung surface. The Hanks' Balanced Salt Solution was then removed from the tube and discarded. One ml of CHO 2× Lysis Buffer (containing 1 mM phenylmethanesulfonyl fluoride, 0.25M Tris(hydroxymethyl)aminomethane hydrochloride, pH adjusted to 8.0 using PH/mV/Thermometer and Sodium Hydroxide pellets, 0.05M sodium chloride, 0.5% Nonidet® P 40 Substitute solution, 0.5% Sodium deoxycholate) was added to each set of lungs. Holding the Vacutainer® steady, a Variable Speed Homogenizer was lowered into the lung solution with the blades sitting on top of the lung mass. The Variable speed Homogenizer was applied for about 20 to 30 seconds to completely disrupt lung tissue. Care was taken to avoid over-homogenization or the generation of heat that may degrade the antibody. The homogenized lung tissue solution was transferred to a Falcon® Blue MaX™ 15 ml Polystyrene Plastic Conical tube for sonication. The material was sonicated 3 times for about 30 seconds on ice. The homogenized, sonicated lung tissue suspension was then transferred into a Microcentrifuge Tubes and spun for 15 minutes in a Marathon Micro H Fisher Scientific microcentrifuge. Afterwards, the supernatant was removed and stored at −20° C. until a protein and antibody assay. The pellet was discarded.

6.4.1.2 Assay Standards

The assay for total protein was conducted using the BCA (bicinchoninic acid) reagent (Pierce Cat #23225).

Assay standards were created by taking lung tissue from a untreated cotton rat (not receiving antibody), lysing as described above, dividing the total recovered into 5 vials, and spiking known amounts (300 μg/ml, 30 μg/ml, 3 μg/ml and 300 ng/ml, respectively) of the RSV MAB 48 into 4 vials. No MAB was added to the remaining one vial for negative control. Each of the spiked samples and zero control material were diluted with ELISA sample buffer creating log dilutions starting at a 1:100 through 1:10⁵.

6.4.1.3 ELISA Assay

Zymed Rabbit anti mouse IgG2a stock (500 μg/ml, catalog # 61-0200, lot # 20168583) was diluted to 3 μg/ml with carbonate coating buffer (Sigma). Wells of 96-well plate (inner 60 only) were coated with 100 μl of 3 μg/ml Zymed rabbit anti mouse IgG2a carbonate solution, covered and placed in CO₂ incubator for 1 hour. Coating solution was discarded from wells, and the plate was slapped against paper towels to remove residual coating solution. Non-specific sites were blocked with 250 μl of 5% dry milk powder in PBS/Tween 20 (Sigma P3563). The plate was covered with parafilm and incubated for 2 hours in CO₂ incubator (37° C.). The blocking solution was discarded, and the plate washed 3 times with PBS/Tween 20, then slapped on paper towels to remove any residual wash. Samples and RSV 48.4.1 controls (primary antibody) were diluted appropriately to fit on the standard curve. The standard curve was comprised of RSV MAB 48 at 300 ng/100 μl well, 100 ng/100 μl well, 30 ng/100 μl well, 10 ng/100 μl well, 3 ng/100 μl well, 0.3 ng/100 μl well, and a blank. Samples were diluted in 0.5% dry milk in PBS/Tween 20, with 2 repetitions for each dilution and 100 μl to each well. The plates were then incubated for 1 hour in a CO₂ incubator.

The primary antibody solution was discarded, and the plate washed 3 times with PBS/Tween-20 and slapped on paper towels to remove residual wash. 100 μl of HRP conjugate was then added to each well (goat anti-mouse conjugate pool). To prepare HRP conjugate pool, 2 μl of anti-IgG2a (Southern Biotech) and 4 μl of anti-IgG (Sigma) were added to 30 ml of PBS/Tween-20 solution containing 0.5% w/v milk powder. One hundred microliters of the solution was added to each well. Covered plate was incubated for 1 hour in CO₂ incubator (37° C.). The secondary antibody solution was then discarded, and the plate washed 4 times with PBS/Tween 20 and slapped on paper towels to remove residual wash. One hundred microliters of TMB (Sigma T8665) substrate was added to each well and allowed to develop in the dark for 30 minutes. Two hundred microliters of 0.5 M H₂SO₄ was added to each well, and the absorbance was read at 450 nanometers.

The actual weight of antibody was determined by applying the forecast function (in Microsoft Excel) to the raw OD values. The forecast function calculates or predicts a future value by using existing values. For example, the predicted value is a y-value for a given x-value. The known values are existing x-values and y-values and the new value is predicted by using linear regression.

6.4.1.4 Model Antibody

The antibody used in this single route ID vs. IM study (RSV MAB 48) is a murine IgG2a. RSV MAB 48 recognizes the fusion protein and has been shown to block infection in tissue culture experiments. The antibody was propagated in ascites, purified with a Zymed Protein-A Column, dialyzed in 1/10 PBS to remove as much excipient as possible without losing antibody solubility.

6.4.1.5 Dosages

Each animal received an injection of 15 mg/kg body weight. Considering that the excipient represented almost half of the weight, the cotton rats received an actual antibody dose of about 7.5 mg/kg.

A sample calculation for both IM and ID injections is shown below:

Cotton rat weight=98.9 grams;

To administer 75 μl of antibody solution at 15 mg/kg, 1.48 mg/75 ul concentration is needed

To include syringe waste, 3.94 mg is placed into 200 ul, and 75 μl was administered.

6.4.1.6 Delivery Devices and Methods

IM and ID injections were performed using a 30 G BD needle. The IM injection was performed by pinching the rear leg muscle, creating depth. With the needle at an angle so the bevel could be buried in the muscle, the IM injection volume was delivered, and was palpable in the muscle. The ID injection was performed by entering at the most shallow angle possible, then turning the bevel up before injection creating a “bleb”.

6.4.2 Results

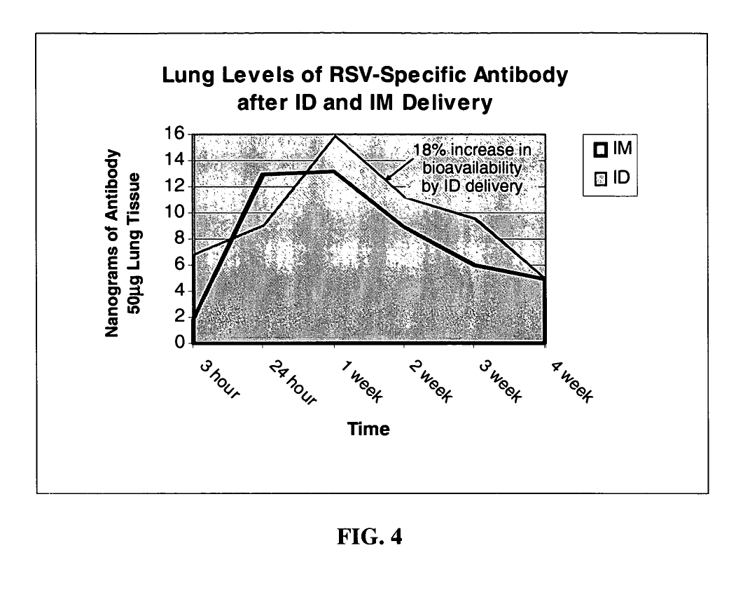

From the assay of lung tissues at 3 hours, 24 hours, 7, 14, 21 and 28 days post-injection, an enhancement in organ antibody for lung tissues obtained from ID-administered animals, as compared to those obtained from IM-administered animals, was observed from the first assay, i.e., 3 hours post-injection (FIG. 4). The last time point where this enhancement was observed was at 21 days post-injection. At 28 days post-injection, no difference between organ antibodies were observed between ID- and IM-administered animals.

The largest percent difference was recorded at 3 hours post-injection where the ID-delivered animals showed 350% more antibody in their lungs than the IM-delivered animals. The overall enhancement, totaling all assays from six time points, was about 18% (about 57 ng for ID-administered animals v. about 48 ng for IM-administered animals). In addition, the 3-hour ID samples indicated a faster onset of antibody in the lungs, and the 1-week ID samples indicated a higher peak level (Cₘₐₓ) of antibody in the lungs.

This data demonstrates ID delivery is capable of achieving a higher level of antibody in the target tissue (lung), without an increase in the amount of antibody administered. Therefore, less antibody needs to be delivered intradermally, compared to intramuscularly, to achieve a similar level of protection. In addition to cost advantages, if less material is required to maintain the target trough value in the lungs, then the risk of eliciting undesirable side effects such as Human Anti-Mouse Antibodies (HAMA) decreases. Without being limited by a particular theory, the enhanced bioavailability may be due to targeting a tissue site for injection that promotes better absorption of the active compound and/or minimizes undesired degradation of the drug.

6.5 Split Dose Dual Route Study

6.5.1 Experimental Design

Preliminary in-house bioavailability studies conducted in cotton rats proved direct intranasal application of antibody could lead to higher peak levels of antibody in lung tissue. This observation led to the formation of a split-dose dual route strategy for obtaining both treatment and prophylactic levels of antibody. This study was performed in Balb/c mice (Grp n=5).

A series of preparatory experiments were conducted first to determine the optimal IN delivery volume and subsequent sampling times. Collection of the lung material was performed as described in Section 6.4, except for the timing. Since the animals in this study received a different RSV Specific MAB (Palivizumab), the ELISA of the Lung assay differed from the method described in Section 6.4. In these preliminary experiments, groups of Balb/c mice received the Synagis Palivizumab at 15 mg/kg body weight in three separate IN dosing volumes. After administration, lung tissue was collected at 1, 3 and 5 hours. Five mice were used per test and control groups. The total lung tissue from each animal was collected, homogenized in a lysis buffer, clarified by centrifugation and the supernatant placed in an ELISA against an immobilized RSV-FP peptide. Lung homogenates within a test or control group were pooled for assay.

6.5.2 ELISA Procedures

ELISA was performed using the following exemplary ELISA procedures:

1. Remove 1 mg/ml stock of RSV F 64-mer peptide from Freezer. Dilute stock to 10 μg/ml with carbonate coating buffer (Sigma);

2. Coat wells of 96-well plate (inner 60 only) with 100 μl of 10 μg/ml RSV FP 64-mer. Cover and place in CO2 incubator for 1 hour;

3. Discard coating from wells into sink and slap plate against paper towels to remove residual coating solution. Block non-specific sites with 250 μl of 5% dry milk powder in PBS/Tween 20 (Sigma P3563). Cover plate with parafilm and incubate for 2 hours in CO2 incubator (37° C.);

4. Discard blocking solution into sink and wash plate 3 times with PBS/Tween20. Slap plate on paper towels to remove residual wash;

5. Dilute samples to fit on standard curve. For standard curve, use Palivizumab at 300 ng/100 μl(well), 100 ng/well, 30 ng/well, 10 ng/well, 3 ng/well, 1 ng/well, 0.3 ng/well and blank. Dilute samples in 0.5% dry milk in PBS-Tween 20. Perform 2 reps for each dilution. Place 100 μl to each well. Incubate plate for 1 hour in CO2 incubator;

6. Discard primary antibody solution into sink and wash plate 3 times with PBS/Tween-20. Slap plate on paper towels to remove residual wash;

7. Add 100 μl of HRP conjugate to each well (goat anti human Ig). To prepare HRP conjugate working stock, add 2 μl of anti-Human Ig (Promega) to 30 ml of a PBS/Tween-20 solution containing 0.5% w/v milk powder. Vortex and add 100 μl to each well. Incubate covered plate for 1 hour in CO2 incubator (37° C.);

8. Discard secondary antibody solution into sink and wash plate 4 times with BS/Tween 20. Slap plate on paper towels to remove residual wash;

9. Add 100 μl of TMB (Sigma T8665) substrate to each well and allow to develop in dark for 30 minutes; and

10. Add 200 μl of 0.5 M H2SO4 to each well and read absorbance at 450 nanometers.

The actual weight of antibody was determined by applying the forecast function (in Microsoft Excel) to the raw OD values.

6.5.3 Delivery Devices and Methods

IM injections were again performed using a 30 G BD needle (reorder #305106). The IM injection site was prepared by pinching the rear leg muscle to create depth. The needle was angled so the bevel could be adequately buried in the muscle. The injection volume was delivered and was palpable in the muscle.

IN doses were administered with a P200 Ranin Pipetman fixed with a disposable tip.

6.5.4 Results

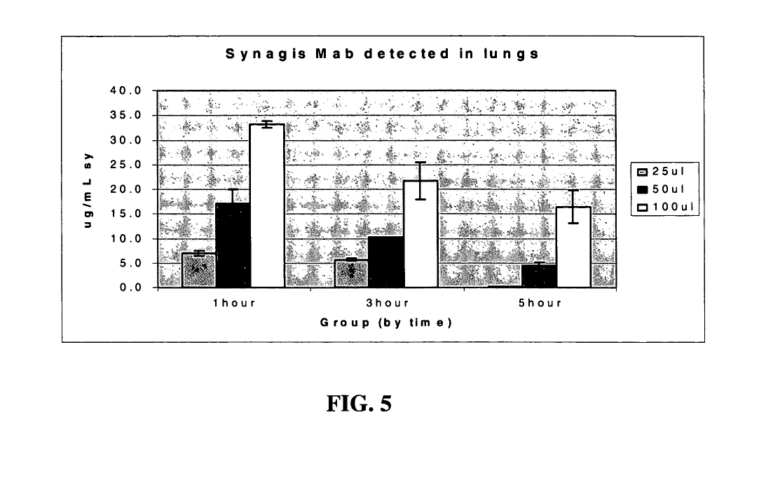

FIG. 5 shows a steady increase in lung antibody concentration coinciding with an increase in intranasal dosing volume. IN-delivered antibody was short lived. However, the peak concentration of antibody detected in the 100 μl group is at least 2 times greater than levels recorded with any other delivery route. These findings were consistent with earlier observations made from cotton rat studies. While the IN delivered antibody is cleared quickly, antibodies with normal affinity need only to be present for a period of minutes given the antibody concentration and organ volume involved in this study.

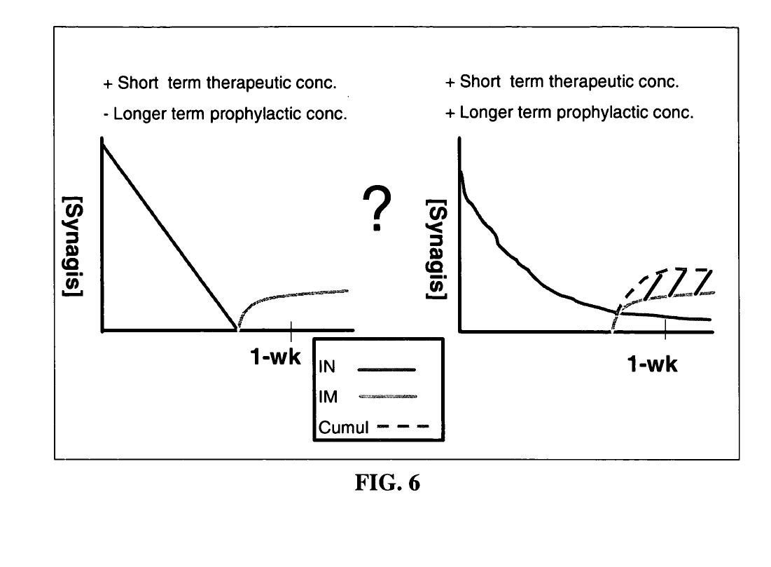

Utilizing the findings above, the bioavailability of Palivizumab delivered IM was compared to a dose divided among IN and IM routes. Balb/c (Grp n=5) mice received the Synagis Palivizumab at 15 mg/kg body weight. All IM and IN injection volumes were 75 μl. Lung tissue was collected at 1-hr post administration and again at 1-wk. The total lung tissue from each animal was collected, homogenized in a lysis buffer and clarified by centrifugation as described in Section 6.4, above. A BCA protein assay was performed on all clarified homogenates allowing test and control tissue to be compared on an equal protein basis. The clarified lung homogenates from each group were pooled and placed in ELISA plate wells coated with RSV-FP peptide. FIG. 6 provides examples of a desired and undesired outcome.

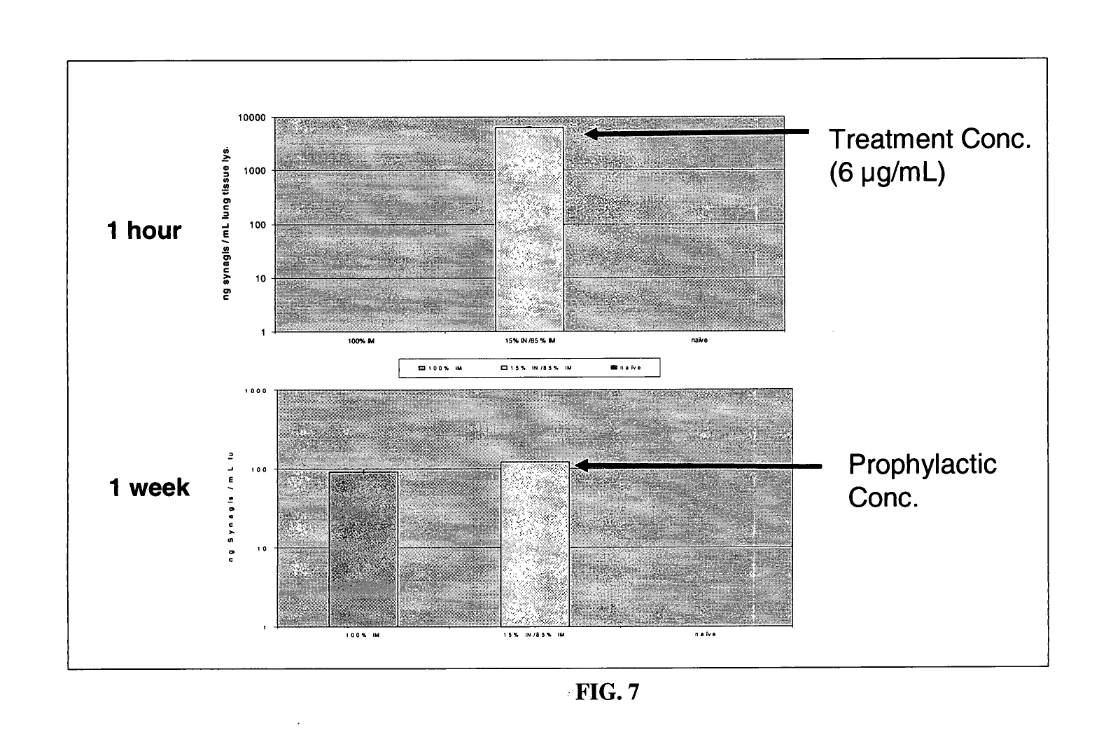

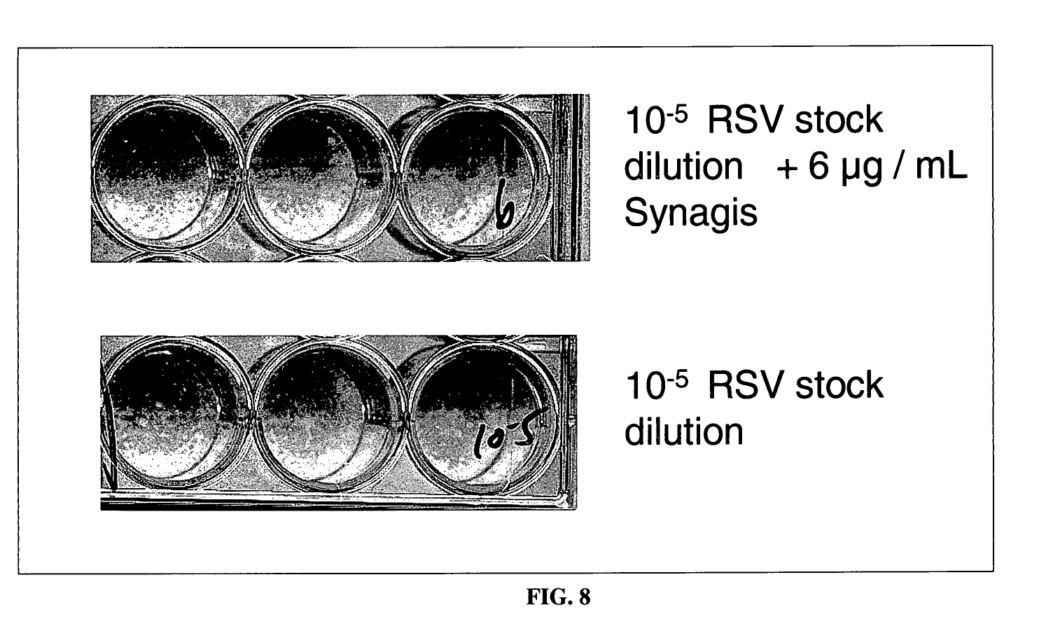

FIG. 7 shows a split dose of 15% IN/85% IM leading to an immediate-substantial level of lung antibody without any reduction in the prophylactic level that must exist at 1-week. Previously, it was determined that a competitive level of antibody at 1-week is a reliable indicator of levels persisting at 1-month. The amount of antibody found in lung tissue collected from the IN/IM group coincides with concentrations of antibody that neutralize infection when assayed in vitro. In contrast, the standard IM dose did not lead to detectable quantities of antibody at 1-hour. At 1-week the level of lung antibody in IM and IN/IM groups was comparable. FIG. 8 shows a 6 μg/ml concentration of antibody that is capable of blocking high numbers of virus in vitro.

6.5.4.1 One Hour Post-Administration

As shown in FIG. 4, the split-dose dual route delivery strategy led to a treatment concentration of Synagis-Palivizimab in lung tissue that was >6 μg/ml of clarified lung tissue homogenate. In contrast, the standard IM delivery of Synagis-Palivizimab did not lead to any detectable antibody in lung homogenates.

6.5.4.2 One Week Post-Administration

The split-route delivery strategy led to a concentration of Synagis-Palivizimab in lung tissue that was >100 ng/ml of clarified lung tissue homogenate (the prophylactic level of antibody typically achieved with standard IM delivery).

the dual route strategy achieves a treatment level of antibody in lung tissue without suffering loses to the longer-term prophylactic level. All achieved with the standard FDA approved dose. RSV disease specific antivirals and anti-inflammatory drugs may be given at the same time and by the same split-dose dual route manner.

6.6 Virus Challenge

In a follow up study, the inventors demonstrated that 15% of the standard Palivizumab dose delivered IN could resolve an existing infection. Mice were challenged with 1.3×10⁵ plaque forming units. Three hours after infection a test group was treated with a single dose of Palivizumab IN (˜2.25 mg/Kg) and a control group received saline. Three days after infection, lung tissue was collected and homogenized. The subsequent homogenates were dispensed onto Hep-2 monolayers and the cultures were monitored for evidence of viable virus. The total homogenate volume recovered from each animal was approximately 1.5 to 2 mls and the amount placed in each test well was 100 ul. The table below shows Synagis IN resolved the infection as no plaque forming units were observed.

| | Lung Tissue Collected @ 72 Hours |
|---|---|
| Treatment | Post Infection (assayed straight) |
| Animals Treated with IN | 29 PFU/Well |
| Saline | |
| Animals Treated with IN | No PFU Observed |
| Synagis (15% of Dose) | |

1. A method for treating a cancer in a human subject in need thereof, comprising delivering at least one therapeutic agent to an intradermal compartment of the human subject's skin, wherein the therapeutic agent results in a greater reduction in the growth of the tumor as compared to when the agent is delivered by a route other than intradermal delivery.

2. A method for treating a cancer in a human subject comprising delivering at least one therapeutic agent to an intradermal compartment of a human subject's skin, wherein the agent results in an increase in the median life span of the human subject as compared to when the agent is delivered by a route other than intradermal delivery.

3. The method of claim 1 or 2, wherein the route other than intradermal delivery is subcutaneous delivery.

4. The method of claim 1 or 2, wherein the route other than intradermal delivery is intramuscular delivery.

5. The method of claim 1 or 2, wherein the route other than intradermal delivery is intravenous delivery.

6. The method of claim 1 or 2, wherein the route other than intradermal delivery is epidermal delivery.

7. The method of claim 1 or 2, wherein the cancer is selected from the group consisting of lymphoma, leukemia, breast cancer, melanoma, lung cancer, renal cancer and colorectal cancer.

8. A method for administration of at least one therapeutic agent to a human subject, comprising delivering the agent into the intradermal compartment of the human subject's skin so that the agent has a higher tissue bioavailability in a particular tissue as compared to when the agent is delivered by a route other than intradermal delivery.

9. The method of claim 8 wherein the therapeutic agent is administered for the treatment of a disease selected from the group consisting of cancer, metastesis of cancer, tumor growth or infectious disease.

10. A method for administration of at least one therapeutic agent to a human subject for the prevention of a disease, comprising delivering the agent into the intradermal compartment of the human subject's skin so that the agent has a higher tissue bioavailability in a particular tissue as compared to when the agent is delivered by a route other than intradermal delivery.

11. The method of claim 10 wherein the disease prevented is selected from the group consisting of cancer, metastesis of cancer, tumor growth or infectious disease.

12. A method for administration of at least one therapeutic agent to a human subject for the delay of the onset or the progression of a disease state, comprising delivering the agent into the intradermal compartment of the human subject's skin so that the agent has a higher tissue bioavailability in a particular tissue as compared to when the agent is delivered by a route other than intradermal delivery.

13. The method of claim 12 wherein the disease state is selected from the group consisting of cancer, metastesis of cancer, tumor growth or infectious disease.

14. The method of claim 1, 2, 8, 10, or 12 wherein the agent is selected from the group consisting of anti-cancer agents, anti-neoplastic agents, and chemotherapeutic agents.

15. The method of claim 1, 2, 8, 10, or 12 wherein the agent is selected from the group consisting of antibodies, vaccines and cell therapies.

16. The method of claim 15 wherein the antibody is selected from the group consisting of a preventative antibody, a polyclonal antibody, a monoclonal antibody, a murine antibody, a human antibody, a chimeric antibody or an antibody fragment.

17. The method of claim 1, 2, 8, 10, or 12 wherein the agent is selected from the group consisting of angiogenesis inhibitors, cytokines or chemokines.

18. The method of claim 17 wherein the cytokine is interleukin or interferon.

19. The method of claim 18, wherein the interleukin is interleukin-12.

20. The method of claim 1, 2, 8, 10 or 12, wherein the agent is delivered by a needle or a cannula.

21. The method of claim 1, 2, 8, 10 or 12, wherein the outlet of the needle or the cannula is inserted to a depth of about 300 um to about 3 mm.

22. The method of claim 1, 2, 8, 10 or 12, wherein the needle or cannula is 30-36 gauge.

23. The method of claim 1, 2, 8, 10 or 12, wherein the needle or cannula is 31-34 gauge.

24. The method of claim 1, 2, 8, 10 or 12, wherein the agent is delivered through at least one small gauge hollow needle having an outlet with an exposed height between 0 and 1 mm, said outlet being inserted into the skin to a depth of between 0.3 mm and 2 mm, such that delivery of the substance occurs at a depth between 0.3 mm and 2 mm.

25. The method of claim 8, 10 or 12, wherein the particular tissue is selected from the group consisting of lymphatic tissue, mucosal tissue, lymph nodes, skin tissue, reproductive tissue, cervical tissue, vaginal tissue, lung, spleen, colon, thymus, bone marrow, haemolymphoid tissue, and lymphoid tissue.

26. The method of claim 25 wherein the lymphoid tissue is selected from epithelium-associated lymphoid tissue, mucosa-associated lymphoid tissue, primary lymphoid tissue, secondary lymphoid tissue.

27. The method of claim 8, 10 or 12, wherein about 10 pg to about 30 ng of the agent is accumulated in per 50 ug of the particular tissue.

28. The method of claim 8, 10 or 12, wherein about 10 pg to about 15 ug of the agent is accumulated in per 50 ug of the particular tissue.

29. The method of claim 8, 10 or 12, wherein about 1 cg to about 30 ng of the agent is accumulated in per 50 ug of the particular tissue.

30. A method for treating a cancer in a human subject in need thereof, comprising delivering at least one therapeutic agent to an intradermal compartment of the human subject's skin at a pre-selected dose, wherein the pre-selected dose is reduced by at least one half a fold as compared to the dose delivered by a route other than intradermal delivery.

31. The method of claim 30, wherein the agent is interleukin-12.

32. A method for treating a cancer in a human subject in need thereof, comprising delivering at least one therapeutic agent to an intradermal compartment of the human subject's skin so that the agent has a faster onset compared to when the same agent is delivered by a route other than intradermal delivery.

33. The method of claim 32, wherein the agent is interleukin-12.
